(19) United States
(12) Patent Application Publication
(10) Pub. No.: US 2025/0213666 A1
(43) Pub. Date: Jul. 3, 2025

(54) Engineered Probiotic Delivery System for Anti-SARS-COV-2 Treatment and Immunity Against Viruses

(71) Applicant: University of Cincinnati, Cincinnati, OH (US)

(72) Inventors: Nitin S Kamble, Cincinnati, OH (US); Nalinikanth Kotagiri, West Chester, OH (US)

(73) Assignee: University of Cincinnati, Cincinnati, OH (US)

(21) Appl. No.: 18/852,553

(22) PCT Filed: Mar. 31, 2023
(86) PCT No.: PCT/US23/17189

§ 371 Date: Sep. 30, 2024

Related U.S. Application Data
(60) Provisional application No. 63/326,082, filed on Mar. 31, 2022.

(51) Int. Cl. A61K 39/108; A61K 35/00; A61K 35/741; A61K 39/00; C07K 16/10; C07K 16/46; C12N 15/70

(52) U.S. Cl. CPC A61K 39/0258; A61K 35/741; C07K 16/1003; C07K 16/46; C12N 15/70; A61K 2035/115; A61K 2039/505; A61K 2039/523; A61K 2039/542; A61K 2039/575; C07K 2317/569; C07K 2317/76; C07K 2319/035; C12N 2770/20022; C12N 2770/20034; C12N 2770/20071; C12N 2800/101

(57) ABSTRACT

A novel genetically modified bacterium is disclosed. The bacterium has one or more anti-spike glycoprotein nanobodies on the outer membrane of the bacterium. In one embodiment, one or more of the anti-spike glycoprotein nanobodies have been fused with Intimin. In another embodiment, one or more of the anti-spike glycoprotein nanobodies have been fused with Lpp-OmpA.

### CROSS-REFERENCE TO RELATED APPLICATIONS

This application is a continuation of PCT Application No. PCT/US23/17189 filed Mar. 31, 2023, which claims the benefit of the filing date of U.S. Provisional Application No. 63/326,082, filed on Mar. 31, 2022, the disclosures of which are incorporated by reference herein in their entirety.

### STATEMENT REGARDING FEDERALLY FUNDED RESEARCH OR DEVELOPMENT

This invention was made with government support under GM137321 awarded by National Institutes of Health. The government has certain rights in the invention.

### FIELD OF THE INVENTION

Aspects of the present invention relate generally to systems and methods for delivery of therapeutics into a subject for treatment of various diseases and conditions.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

The pandemic caused by severe acute respiratory syndrome coronavirus 2 (SARS-COV-2) continues to spread with devastating consequences, posing serious unprecedented challenges to patients, healthcare systems, and socioeconomics of science and societies, that thus mandates an innovative scientific approach to contain it. Efforts from the scientific community around the globe are rapidly advanced to develop an effective vaccine. With many COVID-19 vaccines authorized and billions of doses already manufactured and distributed in record time, there's still an urgent need, not only to develop an effective strategy, but also a second-generation vaccine to improve upon the current options. One way is via needle-free administration which could help the vaccine break through distribution bottlenecks and bolster uptake among population afraid of needles.

Historically, vaccination remains the most effective strategy for preventing diseases by substantially reducing the burden of infectious diseases, still there is a growing demand to design safer and novel vaccines and the delivery systems to protect against emerging cryptic, and unaddressed enigmatic diseases. Traditionally, the gold standard of therapeutic drug administration is via the oral delivery of the therapeutics, due to the possibility of self-administration, improved safety, patient compliance, easier manufacturing, and the ease of distribution as compared to the injection-based therapies. The current COVID19 vaccines are however delivered by intramuscular injections, and therefore an oral vaccine has been on strong agenda since the beginning of the pandemic. Protective oral vaccine could transform the landscape of traditional vaccination, eliminating the need for needles, syringes, and the trained vaccinators. However, currently vaccine makers remain reluctant to dispense with the neutralizing antibody response against Spike protein, and the level of protection it offers now appears to be more limited in terms of both the duration of the protection and the breadth of coverage than that of the cellular response. This becomes more apparent as observed with the requirement of third COVID vaccine booster doses. On the contrary, however, stimulation of humoral and cellular immune responses at both systemic and mucosal sites can be achieved using the oral delivery system to establish broader and long-lasting protection.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

The present invention involves a novel genetically modified bacterium having an outer membrane, the bacterium comprising one or more anti-spike glycoprotein nanobodies on the outer membrane of the bacterium. In one embodiment, one or more of the anti-spike glycoprotein nanobodies have been fused with Intimin. In another embodiment, one or more of the anti-spike glycoprotein nanobodies have been fused with Lpp-OmpA. In one embodiment, one or more of the anti-spike glycoprotein nanobodies further comprise one or more restriction sites.

In another embodiment, at least one anti-spike glycoprotein nanobody has a sequence of [SEQ ID NO: 1]. In one embodiment, at least one anti-spike glycoprotein nanobody has a sequence of [SEQ ID NO: 2]. In another embodiment, at least one anti-spike glycoprotein nanobody has a sequence of [SEQ ID NO: 3]. In one embodiment, at least one anti-spike glycoprotein nanobody has a sequence of [SEQ ID NO: 4].

In another embodiment, the genetically modified bacterium is a probiotic. In one embodiment, the bacterium that is modified is E. coli. In another embodiment, the bacterium that is modified is E. coli Nissle 1917. In one embodiment, a pharmaceutical composition is provided that includes the genetically modified bacterium and a pharmaceutically acceptable excipient.

In another embodiment, the present invention involves a novel genetically modified bacterium having an outer membrane, wherein the receptor-binding domain of the spike glycoprotein on SARS-COV-2 (Spike-RBD) is expressed on the outer membrane of bacterium. In one embodiment, the Spike-RBD has been fused with Intimin. In another embodiment, the Spike-RBD has been fused with Lpp-OmpA. In one embodiment, the Spike-RBD has a sequence of [SEQ ID NO: 6]. In another embodiment, the Spike-RBD has a sequence of [SEQ ID NO: 7].

In one embodiment, the genetically modified bacterium is a probiotic. In another embodiment, the bacterium that is modified is E. coli. In one embodiment, the bacterium that is modified is E. coli Nissle 1917. In another embodiment, a pharmaceutical composition is provided including the genetically modified bacterium and a pharmaceutically acceptable excipient.

### BRIEF DESCRIPTION OF THE DRAWINGS

The accompanying drawings, which are incorporated in and constitute a part of this specification, illustrate embodiments of the invention and, together with the general description of the invention given above, and the detailed description given below, serve to explain the principles of the invention. Similar reference numerals are used to indicate similar features throughout the various figures of the drawings.

FIGS. 1A-1H are a series of images showing modular constructs for surface displaying nanobodies on the bacterial cell surface. FIG. 1A shows a schematic design of Intimin-fused nanobodies Ty1, while FIG. 1B shows a schematic design of VHH72. FIG. 1C is a schematic showing the surface display mechanism with pInt-Ty1 and pInt-VHH72 protein expression. FIG. 1D is a western blot analysis. FIGS. 1E and 1F show schematic designs, respectively, of Lpp-OmpA-fused nanobodies Ty1 and VHH72. FIG. 1G shows the surface display mechanism of pLpp-OmpA-Ty1 and pLpp-OmpA-VHH72 protein expression. FIG. 1H is a western blot analysis.

FIGS. 2A-2H are a series of graphs showing that anti-spike nanobody bearing bacteria inhibit Spike-RBD and ACE-2 receptor interaction. FIGS. 2A-2D are graphs showing that functional expression of nanobodies was confirmed with a fluorescence-based assay for nanobody-pInt-Ty1 (FIG. 2A), nanobody-pInt-VHH72 (FIG. 2B), pLpp-OmpA-Ty1 (FIG. 2C), and pLpp-OmpA-VHH72 (FIG. 2D). FIGS. 2E-2H are graphs showing percent Spike-RBD-ACE-2 receptor binding inhibition assay using the nanobodies pInt-Ty1 (FIG. 2E), pInt-VHH72 (FIG. 2F), pLpp-OmpA-Ty1 (FIG. 2G), and pLpp-OmpA-VHH72 (FIG. 2H).

FIGS. 3A-3G are a series of images and graphs showing detection of nanobodies and Pseudo virus neutralization assay using Ty1 nanobodies recovered from the immunized animals. FIG. 3A is a western blot analysis. FIGS. 3B-3G are graphs showing the results of a pseudo virus neutralization assay.

FIGS. 4A-4I are a series of images showing nanobody expression on OMVs. FIG. 4A is a schematic showing OMV formation in E. coli Nissle 1917. OMVs FIG. 4B is an image of an OMV using Transmission Electron Microscopy (TEM). FIG. 4C is a graph showing size distribution by intensity by dynamic light scattering (DLS) using Zetasizer (Malvern). FIGS. 4D-4G are graphs showing OMV size comparison of OMVs collected from pInt-Ty1 (FIG. 4D), pInt-VHH72 (FIG. 4E), pLpp-OmpA-Ty1 (FIG. 4F), and pLpp-OmpA-VHH72 (FIG. 4G) with OMVs collected from WT-EcN. FIG. 4H is a western blot showing successful expression of pInt-Ty1 and pInt-VHH72 nanobodies. FIG. 4I is a western blot showing successful expression of pLpp-OmpA-Ty1 and pLpp-OmpA-VHH72 nanobodies.

FIGS. 5A-5E are a series of images and graphs showing OMVs were detected in the brain and lungs of immunized animals. FIG. 5A is a confocal microscopy image of lungs harvested from a Ty1 subject. FIG. 5B is a confocal microscopy image of lungs harvested from a control bacteria subject. FIG. 5C is a graph showing the results quantified from images 5A and 5B. FIG. 5D is a confocal microscopy image of brain harvested from a Ty1 subject. FIG. 5E is a confocal microscopy image of brain harvested from a control bacteria subject. FIG. 5F is a graph showing the results quantified from images 5D and 5E.

FIGS. 6A-6F are a series of graphs showing that anti-SARS-COV-2 nanobody bearing OMVs inhibit Pseudoviruses expressing Spike protein from binding with ACE2 receptor. Different concentrations of OMVs were measured using a COVID-19 Pseudo virus neutralizing antibody assay (Luciferase) for pInt-Ty1, Lpp-OmpA-Ty1, Empty-CJ23 and control WT-EcN. The concentrations were 22 μg/mL (FIG. 6A), 3 μg/mL (FIG. 6B), and 0.3 μg/mL (FIG. 6C). In addition, different concentrations of OMVs were measured for pInt-VHH72, Lpp-OmpA-VHH72, Empty-CJ23 and control WT-EcN. The concentrations were 22 μg/mL (FIG. 6A), 3 μg/mL (FIG. 6B), and 0.3 μg/mL (FIG. 6C).

FIGS. 7A-7G are a series of images showing EcN expressing surface displayed nanobodies and Spike protein readily aggregate in solution. FIG. 7A is a schematic design of Spike protein surface displayed using Intimin. FIG. 7B is a SDS-PAGE Western blot analysis showing confirmation of the Spike-RBD expression. FIG. 7C is a schematic design of Nb-Spike protein aggregation assay showing bacteria with surface displayed Nb and spike protein forming complexes. FIGS. 7D-7G are graphs showing the results of an aggregation assay using pInt-spike and pInt-Ty1 (FIG. 7D), pInt-VHH72 (FIG. 7E), pLpp-OmpA-Ty1 (FIG. 7F), pLpp-OmpA-VHH72 (FIG. 7G).

FIGS. 8A-8F are a series of graphs and images showing that EcN-Spike induces host immune response and inhibit Pseudoviruses expressing Spike from interacting with ACE2 receptor. Mouse anti-SARS-COV-2 Antibody IgG Titer Serologic Assay was performed using serum samples collected from the mice orally administered with Spike protein expressing bacteria with one (FIG. 8A) and two doses (FIG. 8B). Bacterial residence time was calculated using qPCR (FIG. 8C). IHC analysis on mouse intestines (FIG. 8D) and was quantified with ImageJ analysis (FIG. 8E). Serum samples were used in COVID-19 Pseudovirus neutralizing antibody assay (Luciferase) (Abnova) (FIG. 8F).

FIGS. 9A and 9B are a pair of graphs showing that EcN-Spike induces host immune response and inhibit Pseudo viruses from interacting with ACE2 receptor. A Pseudo virus neutralization assay was performed using serum samples collected from the mice orally administered with Spike protein expressing bacteria every week, for 8 consequent weeks and blood was collected from 1-5^{th} and 8^{th} week. FIG. 9A shows concentration by immunization titer. FIG. 9B shows Pseudovirus-ACE2 neutralization by dose.

FIGS. 10A-10H show an immunohistochemical analysis using mouse intestines. Intestines harvested from the gut of mice orally administered with Spike expressing and control bacteria were analyzed with IHC for CD4+ (FIG. 10A shows the images and FIG. 10B is a graph of the results), CD8+ (FIG. 10C shows the images and FIG. 10D is a graph of the results), CD11b (FIG. 10E shows the images and FIG. 10F is a graph of the results), and IL1β (FIG. 10G shows the images and FIG. 10H is a graph of the results) markers.

FIGS. 11A-11E are a series of images showing plasmid constructs generated for displaying anti-COVID nanobodies on the cell surface. Ty1 and VHH72 nanobodies were surface displayed using Intimin (FIGS. 11A and 11B) and Lpp-OmpA (FIGS. 11C and 11D) surface display signals, respectively. Empty plasmid does not express nanobody (FIG. 11E).

### DEFINITIONS

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The term “engineered”, as used herein, refers to a nucleic acid molecule, protein molecule, complex, substance, or entity that has been artificially designed, produced, prepared, synthesized and/or manufactured. Therefore, the engineered product is a non-naturally occurring product.

As used herein, the term “engineered bacterium” or “engineered bacterial cell” refers to a bacterial cell that has been genetically modified from its native state. For instance, an engineered bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Engineered bacterial cells of the disclosure may comprise exogenous nucleotide sequences on plasmids. Alternatively, recombinant bacterial cells may comprise exogenous nucleotide sequences stably incorporated into their chromosome.

“Probiotic”, as used herein, refers to a live, non-pathogenic microorganism, e.g., a bacterium, which can confer health benefits to a host organism. In some embodiments, the host organism is a mammal. In some embodiments, the host organism is a human. Some species, strains, and/or subtypes of non-pathogenic bacteria are currently recognized as probiotic bacteria. Examples of probiotic bacteria include, but are not limited to, Salmonella typimurium, Listeria monocytogenes, Staphylococcus epidermidis, Bifidobacterium, Bacteroides, Bacillus, Burkholderia cepacia, Propionibacterium, Fusobacterium, Campylobacter jejuni, Lactobacillus acidophilus, Klebsiella, Bacillus coagulans, Enterococcus and Streptococcus, including Streptococcus oralis. The probiotic may be a variant or a mutant strain of bacterium. Non-pathogenic bacteria may be genetically engineered to enhance or improve desired biological properties, e.g., survivability.

A “pharmaceutical composition,” as used herein, refers to a composition comprising an active ingredient (e.g., a bacterial cell, an inducer, a drug, or a detectable compound) with other components such as a physiologically suitable carrier and/or excipient.

As used herein, the term “pharmaceutically acceptable” or “pharmacologically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Moreover, for animal (e.g., human) administration, it will be understood that compositions should meet sterility, pyrogenicity, general safety and purity standards as required by the FDA Office of Biological Standards.

As used herein, the term “pharmaceutically acceptable excipient” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, disintegrating agents, binders, sweetening agents, flavoring agents, perfuming agents, protease inhibitors, plasticizers, emulsifiers, stabilizing agents, viscosity increasing agents, film forming agents, solubilizing agents, surfactants, preservative and antioxidants can also be present in the formulation. The terms such as “excipient”, “carrier”, “pharmaceutically acceptable excipient” or the like are used interchangeably herein.

As used herein, the term “anti-spike glycoprotein nanobody” means a nanobody having an antibody or the fragment thereof on the bacterial surface that exhibits specific binding to spike proteins that are found on coronavirus.

As used herein, the term “nanobody” refers to any single variable domain of heavy immunoglobulin chains.

As used herein, the term “plasmid” refers to a construct composed of genetic material (i.e., nucleic acid).

As used herein, the term “surface display signal” refers to a genetic element that is programmed to be displayed on the bacterial cell surface, (e.g. flagella, pili, Intimin or Lpp-OmpA).

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention, but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

The present invention involves the development of an engineered probiotic as a modular anti-viral platform that can not only be used as an orally administered therapeutic, but it also provides both mucosal and systemic immunity. In one embodiment, E. coli Nissle 1917 (EcN), a probiotic, is engineered to surface display anti-spike nanobodies, and in another, Spike-RBD is surface expressed on the bacterial cell surface. The present invention has found that EcN expressing nanobodies inhibit pseudoviruses expressing spike protein and ACE2 receptor interaction. As shown herein, oral administration of EcN expressing spike protein in the gut, successfully elicits an immune response by means of antibody generation in animals, where the serum antibodies exhibited significant inhibition of pseudovirus and ACE-2 receptor interaction. This was accomplished without losing in situ bacterial viability in the process. This platform has the potential to facilitate development of next generation, designer modular living biotherapeutics for combatting a wide range of emerging cryptic diseases

The human microbiota is a massive, mostly underexplored niche for short-term immunotherapy and long-term adaptive immunity against viruses. Commensal probiotic bacteria have been known to confer protection against pathogenic agents in the GI tract through direct antagonism, competitive exclusion, barrier function, and immune stimulation due to their proximity to Dendritic cells (DCs). These intestinal DC's, also known as Langerhans cells, are among the first cells to encounter pathogens in the GI tract, which upon activation, migrate to the lymph nodes to activate T cells and initiate a protective immune response. Therefore, the immune cells are adequately positioned to interact with the gut bacteria to absorb, process, and present antigens, such as the spike glycoproteins derived from invading viruses. DCs are known to be the strongest functional professional antigen-presenting cells (APCs), which can absorb, process, and present antigens. As the key regulators of innate and adaptive immune responses, DCs are at the center of the immune system and capable of interacting with both B cells and T cells, thereby manipulating the humoral and cellular immune responses. Targeting antigens to DC-specific endocytic receptors has been recently recognized as a promising strategy for designing an effective vaccine that elicits a strong and durable T cell response against diverse types of pathogens. In fact, it was previously analyzed that DCs have the capacity to interact with bacteria and that bacteria can act as “Trojan horses”, delivering heterologous proteins to DCs in a processed form that allows extremely efficient loading of both MHC Class I and Class II molecules.

While commensal bacteria and natural probiotics have some capacity to relay antigens to DCs, it is apparent that their capacity is limited because they lack the arsenal to capture and present the antigens effectively and likely not be able to limit the antigens and viruses to the extracellular space, without losing viability. Moreover, in SARS-COV-2, where lethality is severe, it appears natural defenses are overwhelmed leading to a cascade of reactions manifested in severe morbidity and mortality. The present invention utilizes programmable live bacteria as a unique approach to deliver therapeutic nanobodies as well as viral antigens (spike protein) to improve therapeutic outcomes in viral infections and modulate immune system.

In one embodiment, a probiotic, commensal bacterium E. coli Nissle 1917 (EcN), was utilized for executing this strategy. The rationale behind selecting EcN is the ease of its genetic amenability and wider acceptance as a probiotic. EcN has also been metabolically characterized and shown to be markedly different in comparison to the pathogenic phylogenetic variants of E. coli. EcN synthesizes a dysfunctional O-antigen polymerase due to the mutant wzy gene which impairs the synthesis of virulent liposaccharides, making its cell wall more penetrable and boost immune system-mediated elimination. Absence of virulence factors, such as α-hemolysin and P-fimbrial adhesins further contributes to its non-pathogenic characteristics.

Using constitutive promoters, multiple plasmid constructs for expressing anti-spike nanobodies were generated and successfully displayed on the bacterial cells surface using Intimin and Lpp-OmpA surface anchor proteins. The rationale for evaluating both Lpp-OmpA and Intimin, the later which is relatively longer surface display signal compared to Lpp-OmpA, was to determine the effect of size on surface expressing the nanobodies to enable and enhance their interaction with cognate antigens more effectively in vivo. Following the selection of anti-spike nanobodies and surface displaying strategy, SDS-PAGE western immunoanalysis and quantitative label-free MS proteomics analysis confirmed the expression of nanobodies. EcN-nanobody constructs successfully inhibited spike protein expressing Pseudo viruses from interacting with ACE2 receptor.

Furthermore, similar to mammalian cell-derived exosomes, bacterial OMVs are nanosized vesicles that play a functional role in cellular communication. As OMV production is a natural biological process and OMVs have been reported to release therapeutic payload in situ, by surface expressing the nanobodies we devised a bacterial system capable of distributing anti-spike nanobodies, through OMV mediated translocation to the systemic circulation. Thus, having the potential to alleviate disease onset and buying crucial time for the host system to mount an immune response via humoral and cell-mediated immune systems.

An effective strategy to prevent infection will be by induction of an immunologically strong mucosal barrier at the point of contact between microbes and the host. Unfortunately, the current standards of vaccine technology typically address only those pathogens that have already surpassed a mucosal barrier, and most licensed vaccines are administered either subcutaneously or intramuscularly. The resulting immune response is limited to humoral immunity (e.g., antibody generation) against toxins or pathogens, with limited cellular immunity (e.g., T cell-mediated), and weak protection at the mucosal barriers. On the contrary, vaccination at mucosal surfaces successfully induces cell-mediated immune responses, while simultaneously inducing a systemic antibody response.

Therefore, the present invention engineered EcN to express spike protein. Similar to bacterial cell surface decoration with nanobody, spike-RBD proteins were surface expressed using Intimin signals. Then, a cell aggregation assay was performed between EcN expressing spike-RBD and EcN expressing anti-Spike nanobodies, which revealed significant cell aggregation using both Ty1 and VHH72 nanobodies.

Next, oral administration of EcN-spike in mice gut resulted in generation of systemic immune response through generation of anti-spike antibodies. A dual dose regimen resulted in a prolonged immune response compared to a single dose regimen. Serum collected from immunized mice was also able to inhibit spike protein and ACE2 receptor interaction. Mucosal immunity too was elicited as evidenced by generation of CD4⁺, CD8⁺ T-cells and activated monocytes (CD11b), as well as significantly higher levels of pro-inflammatory cytokine, IL1β, in the intestines orally administered with spike expressing bacteria than those that received control bacteria. The significantly higher expression of T-cells and myeloid cells we observed with IHC analysis are also in concordance with current mRNA vaccines, in which vaccinated recipients show elevated CD4⁺, CD8⁺, monocyte and IL1β responses.

The engineered probiotic essentially serves the dual function of neutralizing viruses directly through surface and OMV expressing nanobodies, by preventing viruses and their surface proteins from binding to ACE-2 receptor; and also provide immunity through expression of spike protein, which interfaces with intestinal DC, assisting in antigen presentation and activation. It has been reported that surface-bound antigen expression uses bacterial chassis as an adjuvant to promote immune cell recognition and uptake. The probiotic platform developed for the present invention is modular in nature, able to integrate any nanobody and viral antigen as a plug-and-play system, allowing the integration of unique nanobodies and antigens against distinct viruses and pathogens. Moreover, the ability of bacteria to thrive and proliferate in situ allows for a sustained response.

One of the key points of the live bacterial vaccine technology is that not only could it be conducive for oral delivery and be room temperature stable, but it could also be mass produced quickly in a cost-effective manner, using scalable and cost-effective manufacturing processes. When it comes to manufacturing, bacteria-based oral vaccines might not require the same high-level biosafety checks compared to systemic vaccines that are based on complete viruses. In depth research on these vaccine candidates will provide valuable insights enabling the development of a room-temperature stable oral formulation, with the goal of delivering the vaccines directly to patients for self-administration thus bypassing the cold-chain supply logistics. This would enable individuals with potential self-administration of vaccine rather than requiring a trained medical professional.

The engineered probiotic platform of the present invention could serve multiple functions in neutralizing initial events after infection as well as provide long-term immunity. This platform could help circumvent limitations associated with the current drug delivery systems, making OMV based nanobody delivery system as a novel and unique carrier for nanobody delivery in-situ. Bacteria and OMVs decorated with the anti-spike nanobody appear as a versatile and robust platform allowing fast production of experimental vaccines and therapeutic agents via a modular plug-and-play procedure.

### EXAMPLES

The following Examples describe the design, construction, and validation of the genetically modified bacterium in accordance with the various aspects of the present invention described herein.

### Example 1: Localization of Surface Display Signal for the Modular Expression of SARS-COV-2 Anti-Spike Glycoprotein Nanobodies

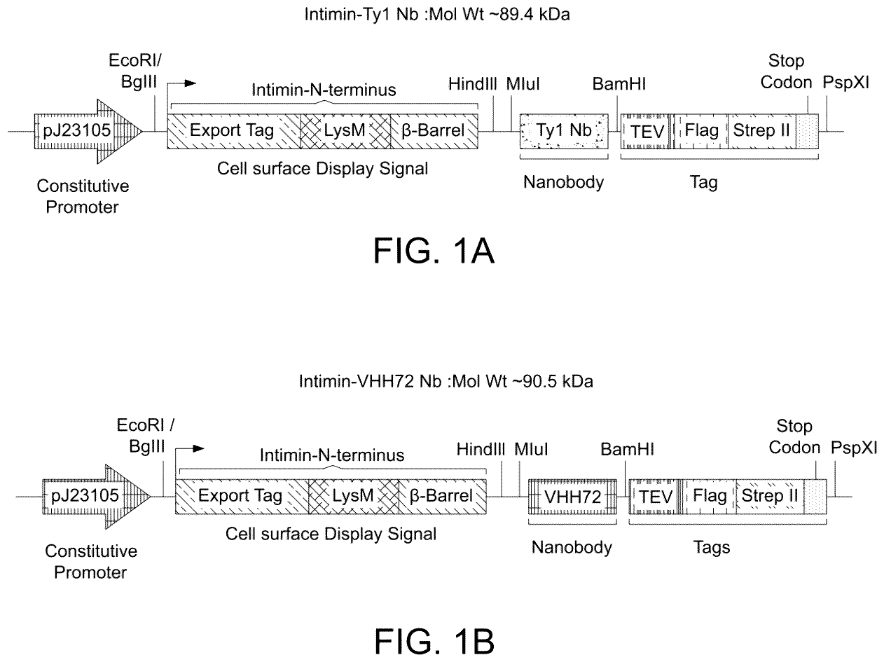

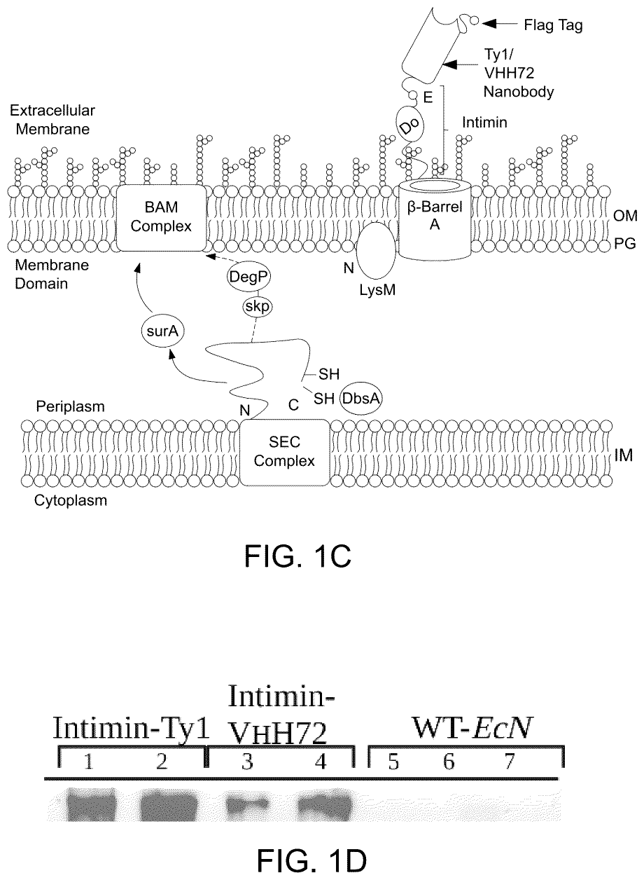

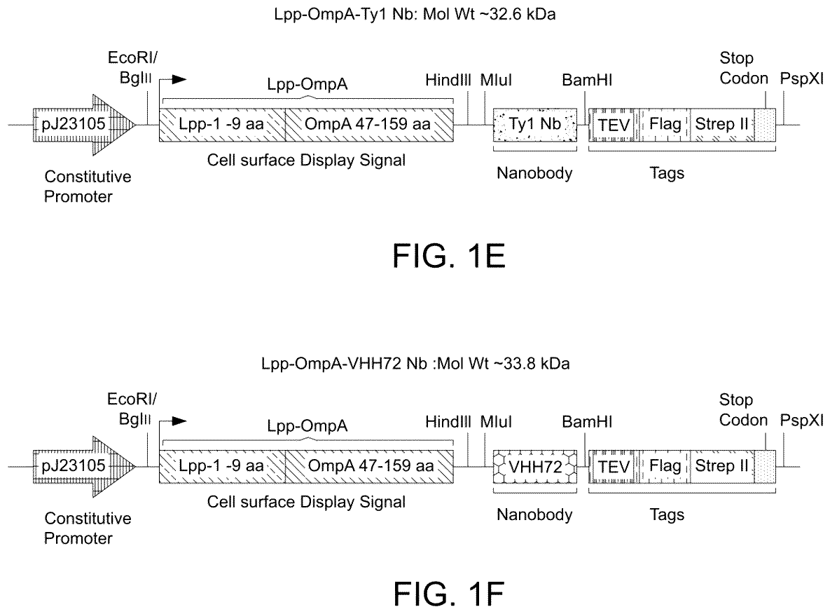

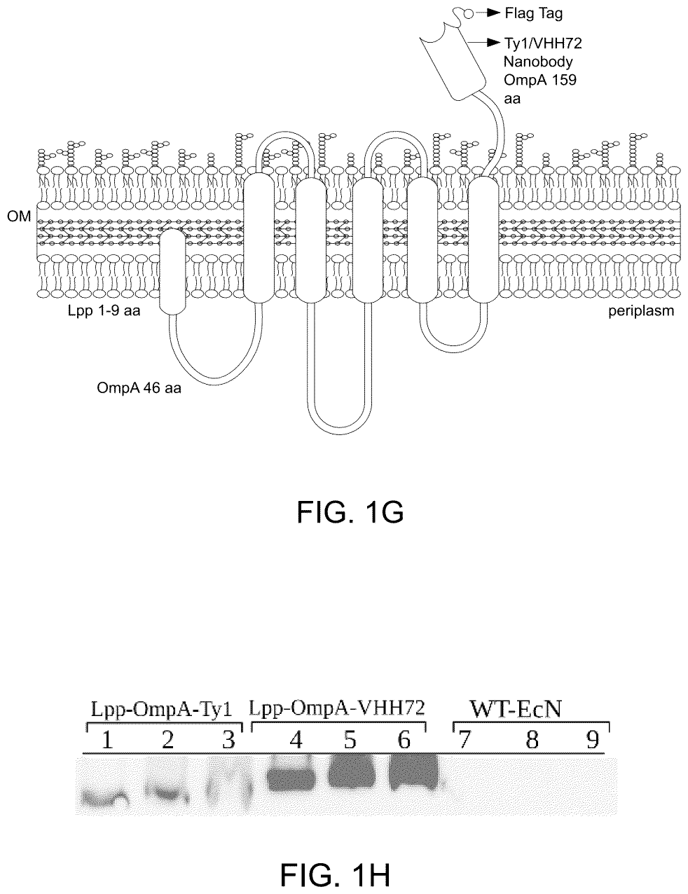

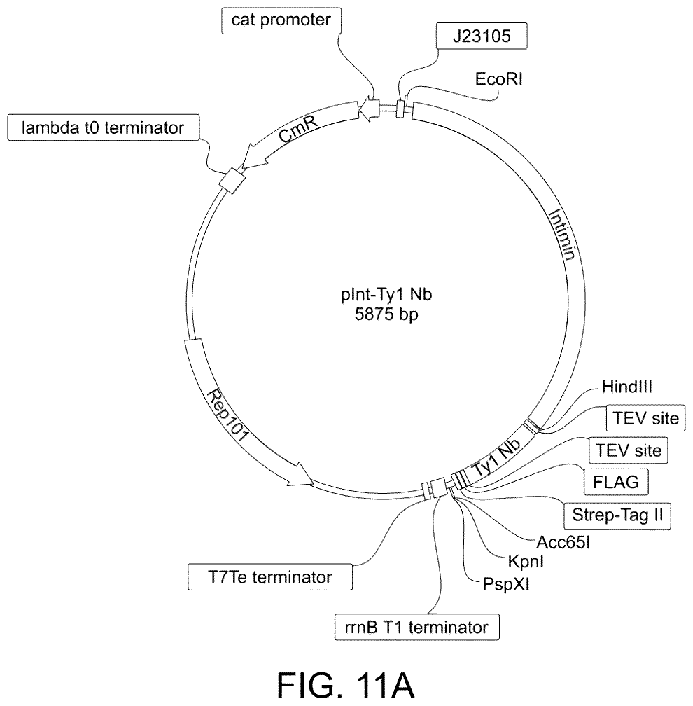

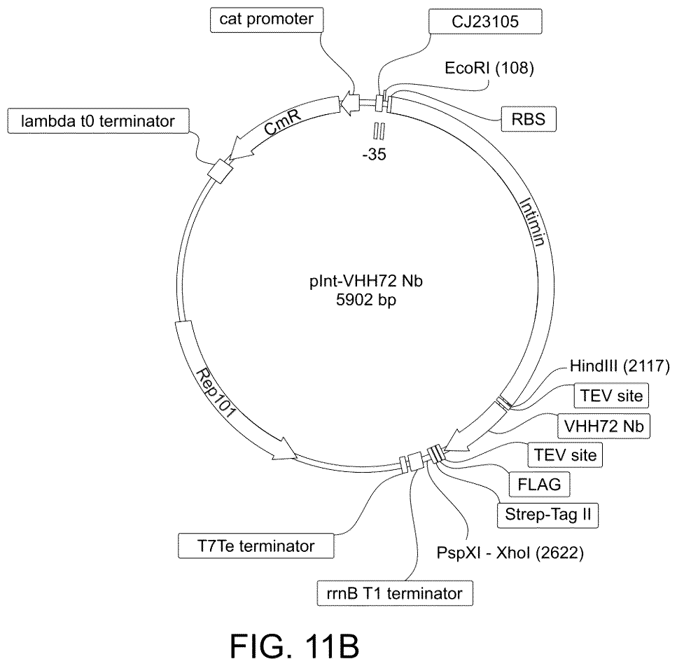

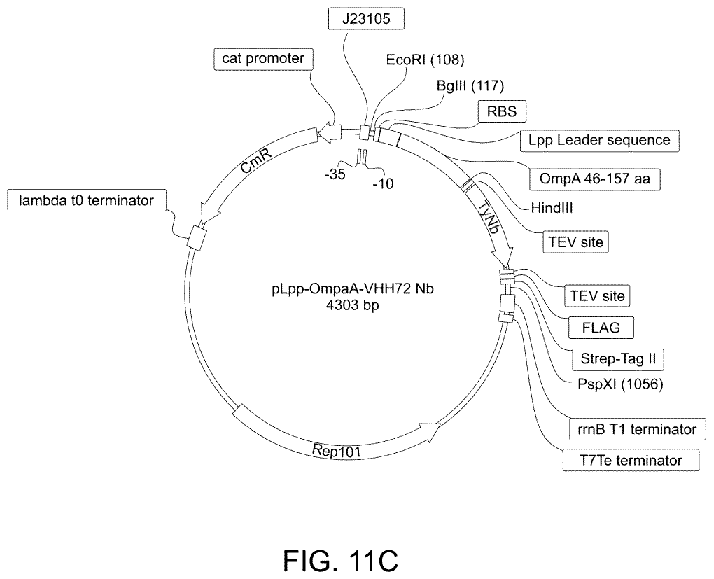

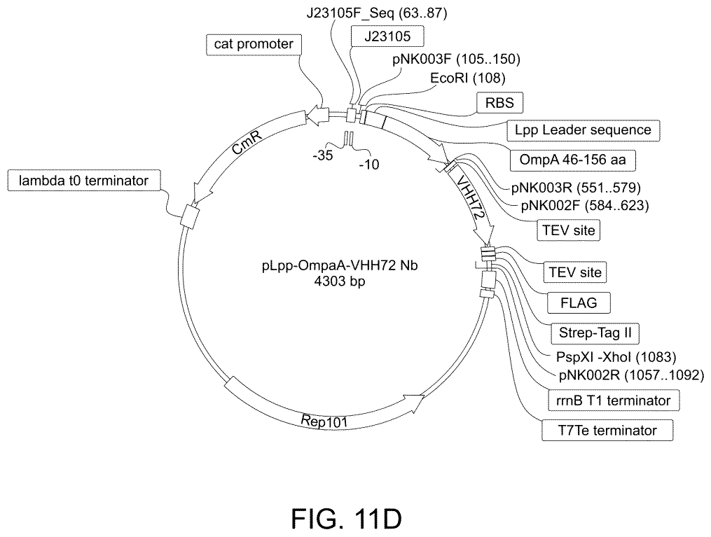

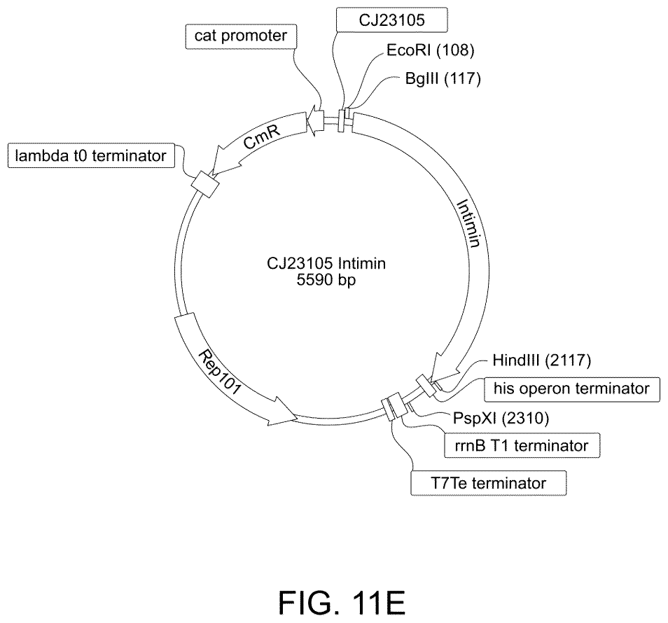

To engineer EcN to express unique, SARS-COV-2-specific nanobodies, and display them on their surface, a synthetic modular vector was designed and tested. This contained genetic elements aimed at displaying the anti-Spike nanobodies on the cell surface, enabling subsequent detection and ultimately purification via insertion of Flag, Step II tag and TEV cleavage sites (FIGS. 11A-11E). These were constructed in a high copy number J23105 plasmid backbone, which contains a moderate strength constitutive promoter to achieve a constant pool of nanobodies. Two nanobody sequences were selected, VHH72 (Salema and Fernández, 2017; Chanier and Chames, 2019) and Ty1 (Hanke et al., 2020a), reported against the receptor-binding domain (RBD) of Spike protein. These nanobody gene sequences were generated de novo utilizing a gene synthesis technology with the flanking BioBrick prefixes/suffixes and were codon optimized. Intimin fused nanobodies-pIntimin-Ty1 (pInt-Ty1) and pIntimin-VHH72 (pInt-VHH72) constructs were then constructed (FIGS. 1A and 1B). Intimin includes a short N-terminal signal sequence (export tag) to dictate its trafficking to the periplasm, a LysM domain for peptidoglycan binding, and a β-barrel for transmembrane insertion (FIG. 1C). The nanobodies were fused to the truncated C-terminus of Intimin. The combination of single-domain structure and the Intimin autotransporters allows the entirety of the cell surface-bound nanobodies to be displayed as a single fusion protein. Finally, restriction sites were incorporated throughout the plasmid backbone to allow modular modification of the construct. These constructs were then transformed into EcN and tested for the expression of anti-spike nanobodies with SDS-PAGE-Western immuno-analysis using HRP-conjugated anti-Flag tag antibody and Mass Spectrometry proteomics analysis. Successful expression of pInt-Ty1 and pInt-VHH72 nanobodies was confirmed with ˜89.4 kDa and ˜90.5 kDa bands (FIG. 1D). Sequence alignment with theoretical sequences of Intimin-Ty1 and Lpp-OmpA-Ty1 was done with trypsin digested peptides obtained from the mass spectrometry analysis of pInt-Ty1 and pLpp-OmpA-Ty1 nanobodies. Blots were developed with anti-Flag tag antibody, using the ChemiDoc™ Imaging system (BioRad), while alignment was performed using Multalign software tool and Biorender.

Additionally, to evaluate the effect of size of the surface tethering protein has on the functional display of nanobodies on the bacterial surface, a second surface display signal was chosen, Lpp-OmpA, that is a considerably smaller tethering protein compared to intimin, to construct pLpp-OmpA-Ty1 and pLpp-OmpA-VHH72 constructs (FIGS. 1E and 1F). The Lpp-OmpA protein is an outer membrane protein expression system which consists of 20 amino acids (aa) of signal sequence, 9 N-terminal amino acids of the lipoprotein (Lpp) and the residual 46-159 aa of the OmpA protein (Mathelié-Guinlet et al., 2020). Lpp is the most abundant protein on the outer membrane, while OmpA domain constitutes 8-stranded, β-barrel to construct an anchor on the outer membrane that provides stable expression of the protein displayed on the outer membrane (FIG. 1G). Similar to Intimin, anti-spike nanobodies (Ty1 and VHH72) were fused to the C-terminus Lpp-OmpA signal, which generated Lpp-OmpA-Ty1 (pLpp-OmpA-Ty1) and Lpp-OmpA-VHH72 (pLpp-OmpA-VHH72) constructs while, an empty plasmid without nanobody was used as a control (FIGS. 11A-11E). Successful expression of pLpp-OmpA-Ty1 and pLpp-OmpA-VHH72 nanobodies was confirmed, with ˜32.6 kDa and ˜33.8 kDa bands, respectively using Western blot analysis (FIG. 1H), with complete absence of any bands in the control sample, EcN expressing empty plasmid-CJ23.

Proteomics analysis was performed to further confirm expression of nanobodies fused with Intimin and Lpp-OmpA anchor proteins. pInt-Ty1 and pLpp-OmpA-Ty1 were used as representatives for the proteomics analysis with Label-free quantitation (LFQ) (Bantscheff et al., 2007; Distler et al., 2016). A total of ˜37 unique peptides were found for pInt-Ty1 as reported in Table 5. For pLpp-OmpA-Ty1, ˜11 unique peptides were found as reported in Table 6. A total ion chromatogram (TIC) was created by summing up intensities of all the mass spectral peaks belonging to the respective scans. Protein abundance was estimated from the counts of tandem MS (MS/MS) spectra attributed to each protein. Finally, principal component analysis (PCA) was performed to reduce the number of dependent variables in the spectral set by replacing groups of intercorrelated variables with single new variables.

### Example 2: Bacterial Surface-Anchored Anti-Spike Nanobodies Inhibit Spike and ACE-2 Receptor Interaction In Vitro

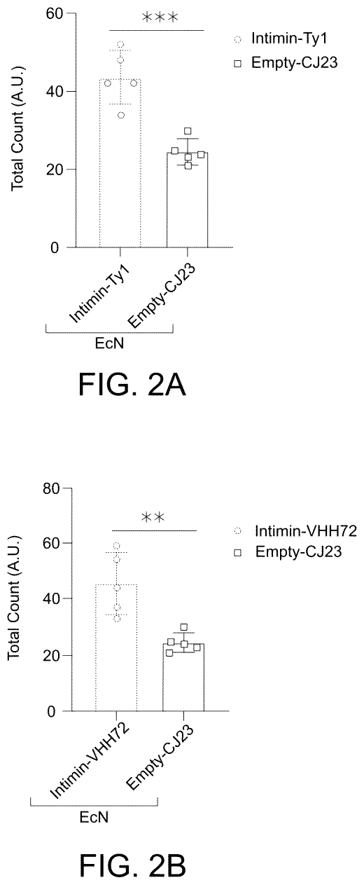

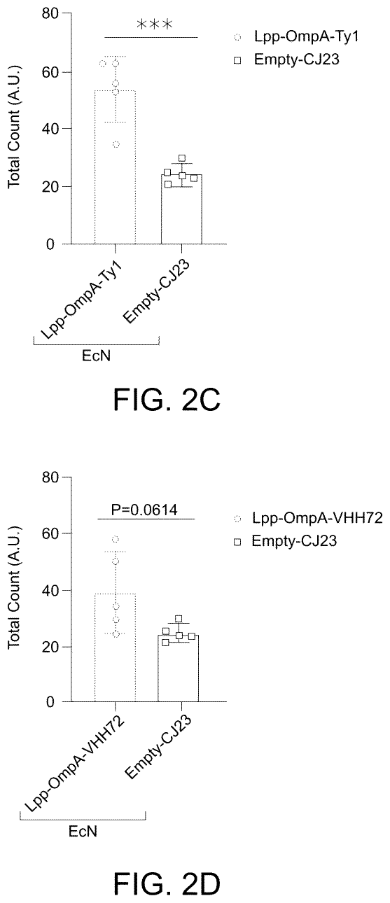

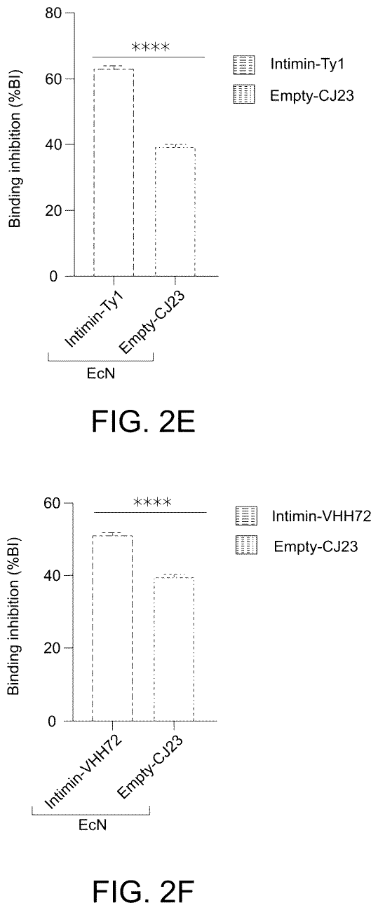

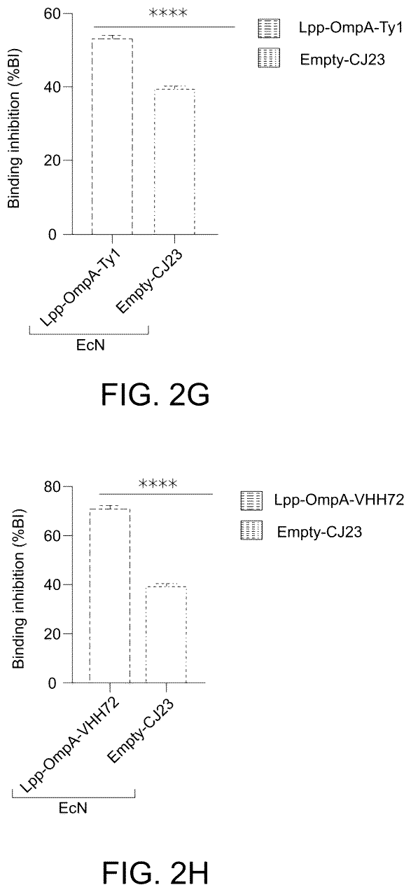

Next, the functional expression of nanobodies was investigated. A fluorescence-based functional assay was performed to evaluate binding of nanobodies expressed on EcN to spike protein. Ty1 and VHH72 nanobodies have been reported in silico to bind to the RBD of the SARS-COVID19 spike protein (Chanier and Chames, 2019; Hanke et al., 2020b; Huo et al., 2020). To confirm the nanobody and spike-RBD interaction, recombinant SARS-COV-2 S (S1+S2) protein was acquired, where nanobodies bind predominantly to the S1 domain of the spike protein. As a control, SARS COV-2 S protein S2 antibody was used, which specifically binds to the S2 domain of the recombinant Spike protein. The sandwich ELISA (Nb-Spike-RBD-S2 antibody) complex was visualized using an AlexaFluor® 647 conjugated anti-mouse IgG2b antibody. It was observed that pInt-Ty1 (FIG. 2A), pInt-VHH72 (FIG. 2B) and pLpp-OmpA-Ty1 (FIG. 2C) bearing EcN have higher binding affinity to Spike protein compared to the control EcN. However, there was no significant difference between pLpp-OmpA-VHH72 bacteria and control EcN with empty plasmid (FIG. 2D). This data demonstrates that three out of four constructs, using Intimin and Lpp-OmpA surface display signals on the bacterial cell membrane, exhibit higher binding affinity to the Spike protein compared to EcN alone.

After confirmation of functional nanobody expression on the bacterial cell surface, it was assessed whether the surface displayed Ty1 and VHH72 nanobodies would inhibit the spike-RBD and ACE-2 receptor interaction. It was observed that all four constructs demonstrated significant binding inhibition of spike-RBD and ACE-2 receptor, compared to EcN alone with empty plasmid. Also, percent binding inhibition was highest with pLpp-OmpA-VHH72>pInt-Ty1>pLpp-OmpA-Ty1>pInt-VHH72 (FIGS. 2E-2H). These results demonstrate that EcN with surface-displayed nanobodies significantly inhibit the recombinant spike protein and ACE-2 receptor interaction.

Referring to FIGS. 2E-2H, a percent Spike-RBD-ACE-2 receptor binding inhibition assay was run using Ty1 nanobody-pInt-Ty1 (FIG. 2E), pInt-VHH72 (FIG. 2F), pLpp-OmpA-Ty1 (FIG. 2G) and pLpp-OmpA-VHH72 nanobody (FIG. 2H) compared to WT-EcN. Images were acquired using fluorescent Microscope (Leica Microsystems) and analysed with ImageJ's Fiji software. Absorbance was measured at 450 nm using WT-EcN as a control. Graphs were plotted using GraphPad Prism 8.0.v, significance was determined using unpaired t²-test, level of significance was set as “*” p<0.05; “**” p<0.01; “***” p<0.001, ns—not significant, (WT-EcN-Wild-type E. coli Nissle 1917).

### Example 3: Nanobodies Recovered from the Blood of Animals Immunized Orally with Nanobody Expressing Bacteria Inhibit Pseudo Viruses and ACE2 Interaction

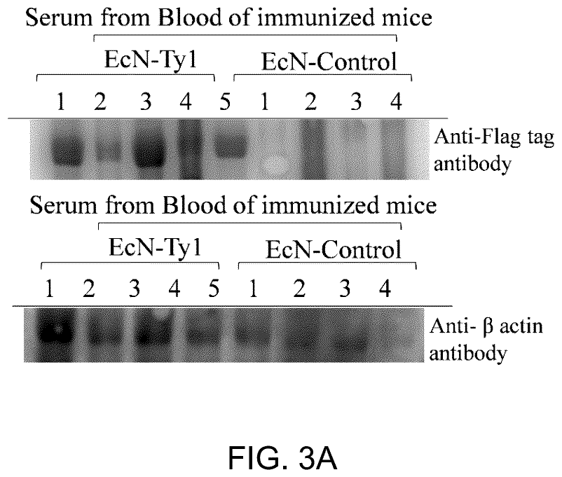

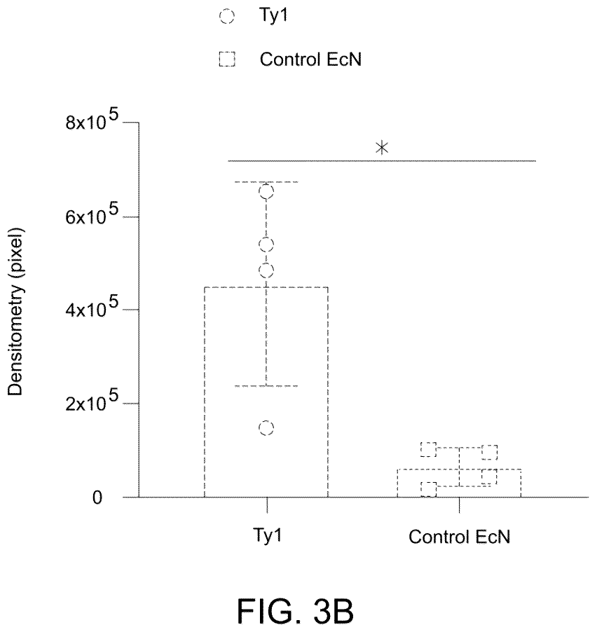

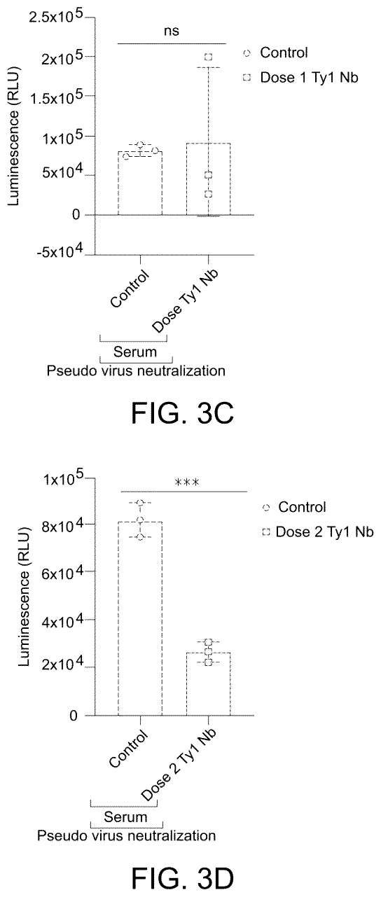

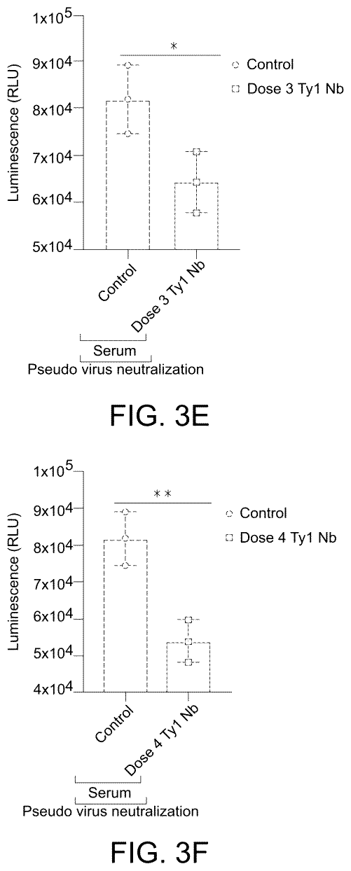

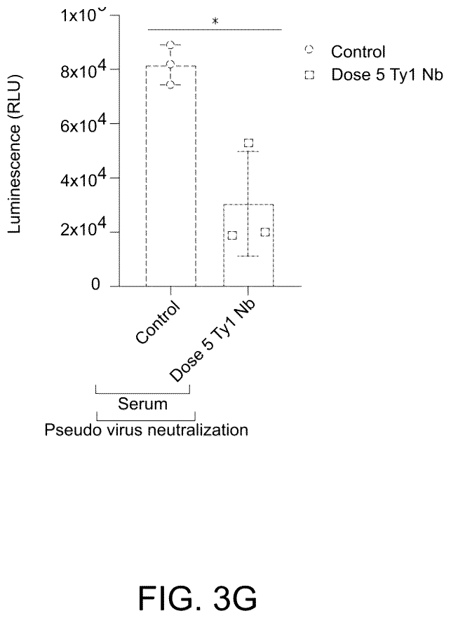

Ty1 nanobody expressing bacteria was orally administered, in parallel with control bacteria, in the mice gut, consequently for 5 days, every day. Blood withdrawn from submandibular vein was subjected to western blot analysis. Ty1 nanobodies were detected using HRP-conjugated anti-flag tag and β-actin antibodies (FIG. 3A) and blot densities were plotted (FIG. 3B), showing significantly higher nanobody expression. Resulting serum samples collected were also used in pseudo virus-ACE2 neutralization assay. Pseudo virus neutralization assay (FIGS. 3B-3G) (Luciferase) was performed using serum samples collected from the mice orally administered 5 days, every day with Ty1 nanobody expressing bacteria, in parallel with control bacteria. Plates were read for Luciferase expression at OD₄₅₀ using EnVision 2102 Multilabel Reader (Perkin Elmer) and graphs were plotted using GraphPad Prism 8.0.v, significance was determined using unpaired t²-test, level of significance was set as “*” p<0.05; “**” p<0.01; “***” p<0.001, ns—not significant.

Significant binding inhibition was found, highest after Dose 2>Dose 4>Dose 3>Dose 5 (FIGS. 3A-3E). However, no significant binding inhibition was found after Dose 1 of Ty1 nanobody administration. This might be suggesting the time required by the nanobodies for reaching into the blood stream and/or the time required for reaching the critical concentration to be able to inhibit the interaction.

### Example 4: Surface Anchored Spike-Glycoprotein Nanobodies were Detected on Secreted Outer Membrane Vesicles (OMVs)

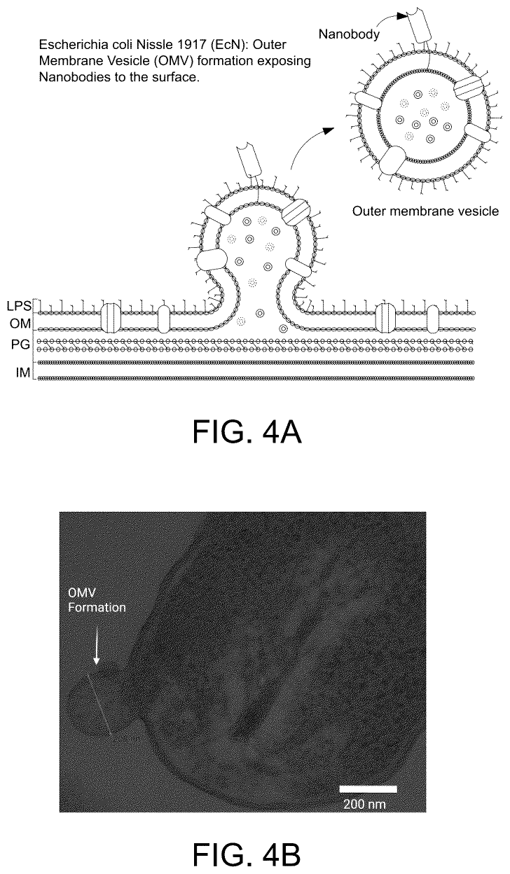

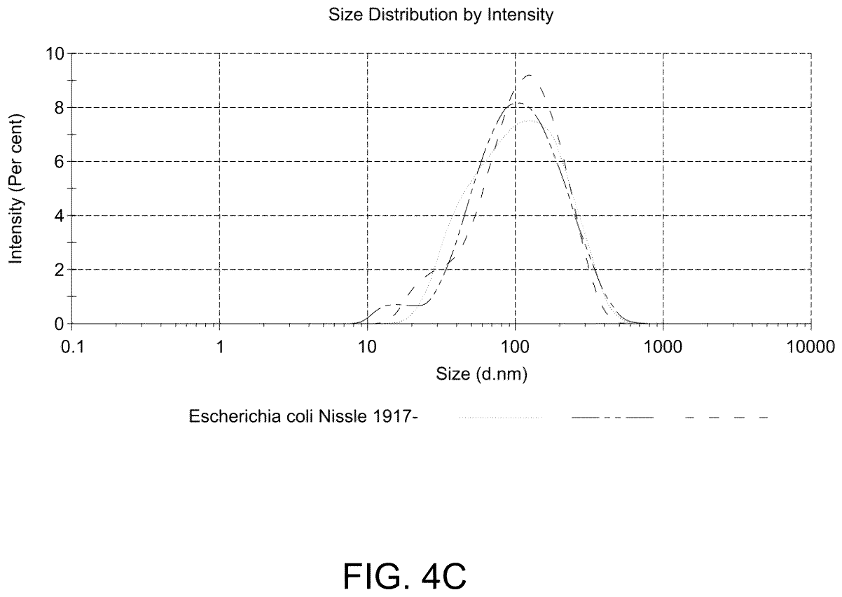

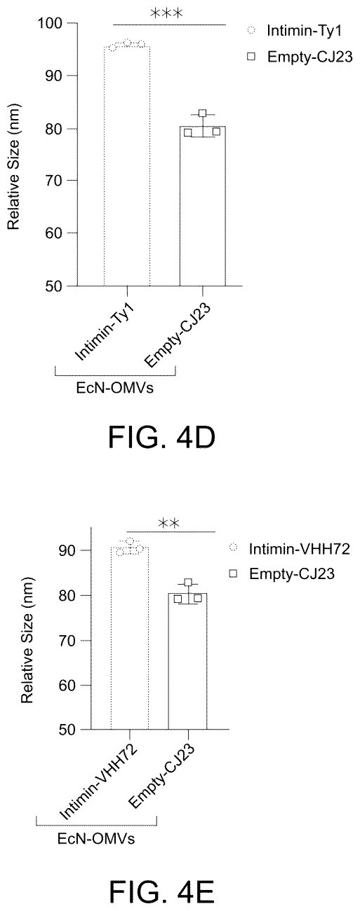

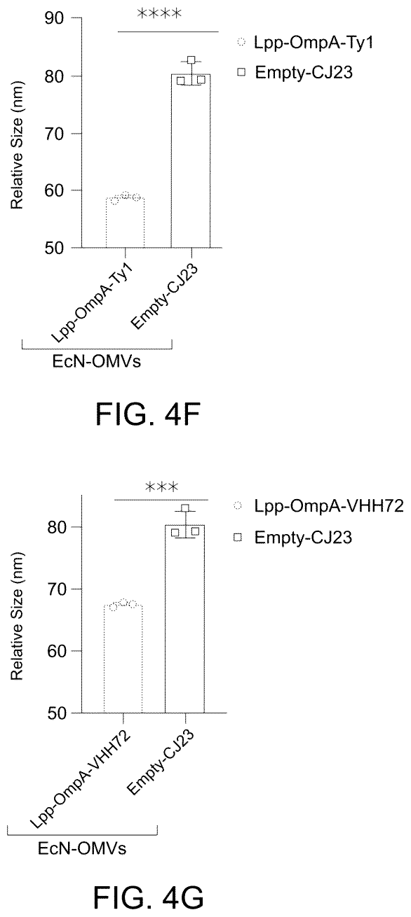

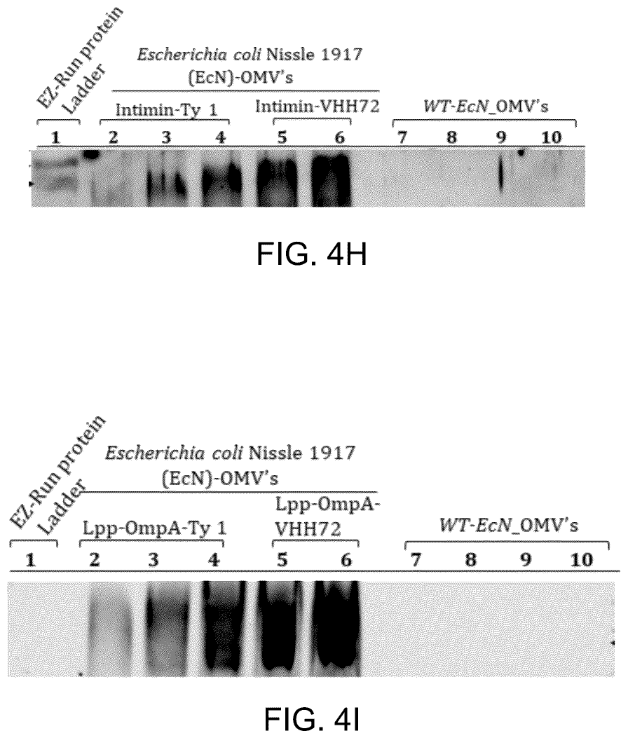

Delivering the nanobody based therapeutics to distal organs where SARS-COV-2 has been reported to infect lungs, kidneys, and testis was explored. Outer membrane vesicles (OMVs) derived from Gram-negative bacteria have been attracting interest in the development of vaccines and therapeutic agents, as compared to live attenuated vaccines (Schwechheimer and Kuehn, 2015; Thomas et al., 2021). OMV production is a natural biological process controlled by a mechanism of blebbing of the outer membrane. OMVs have been reported to release therapeutic payload in situ with the ability to distribute the payload across the intestinal lumen to the dendritic cells (DC) and potentially to the systemic circulation (FIG. 4A) (Girirajan, Campbell and Eichler, 2011; Gujrati et al., 2019; Cheng et al., 2021). These reports suggested that the OMVs secreted by the engineered constructs might contain the Intimin and Lpp-OmpA tethered Ty1 and VHH72 nanobodies. Therefore, OMVs were isolated, purified and phenotypically evaluated via protein measurement with Bicinchoninic acid assay (BCA). The following yields were obtained for WT-EcN OMVs (1.587±0.5 mg/mL) and nanobody bearing OMVs-pInt-Ty1 (5.602±0.5 mg/mL), Int-VHH72 (4.219±0.5 mg/mL), pLpp-OmpA-Ty1 (3.064±0.5 mg/mL) and pLpp-OmpA-VHH72 (1.181±0.5 mg/mL). OMVs of WT-EcN and nanobody bearing bacteria were also analysed with Transmission Electron Microscopy (TEM), which revealed nanosized vesicles with diameters ranging from ˜75 to 400 nm (FIG. 4B). Their size distribution was analyzed with Dynamic light scattering (DLS) (FIG. 4C). The mean vesicle diameter for Intimin anchored nanobodies was slightly larger (90±10 nm) than OMVs of WT-EcN (80±5.0 nm) (FIGS. 4D and 4E). However, the mean vesicle diameter for Lpp-OmpA anchored nanobodies was significantly smaller (60±7.5 nm) than the OMVs carrying Intimin anchored nanobodies, as well as WT-EcN (FIGS. 4F and 4G). They displayed a narrow to moderately (0.2-0.3) polydispersity index (PDI), which is a measure of uniform particle size distribution.

Next, the expression of surface anchored nanobodies was confirmed with SDS-PAGE and Western immunoanalysis using an HRP-conjugated anti-flag tag antibody. Successful expression of pInt-Ty1 and pInt-VHH72 nanobodies, as shown with ˜89.4 kDa and ˜90.5 kDa bands, respectively, were observed, compared to the absence of corresponding bands in the OMVs from WT-EcN (FIG. 4H). Similarly, expression of pLpp-OmpA-Ty1 and pLpp-OmpA-VHH72 nanobodies were detected, as shown with ˜32.6 kDa and ˜33.8 kDa bands, respectively (FIG. 4I). These data suggests that the surface expression of nanobodies on engineered EcN constructs allow the protein to be surface displayed on the OMVs as well.

### Example 5: Ty1 Nanobody Bearing OMVs were Detected in the Lungs and Brains of the Orally Immunized Animals

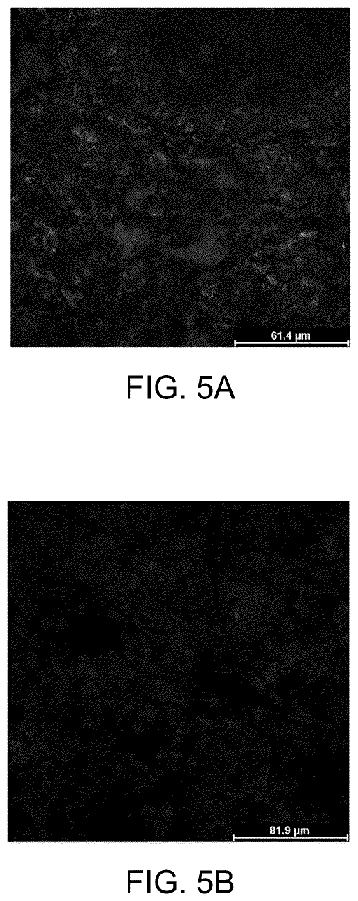

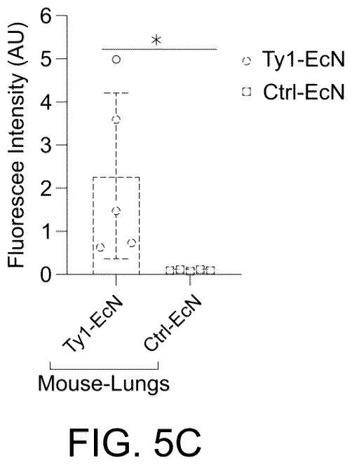

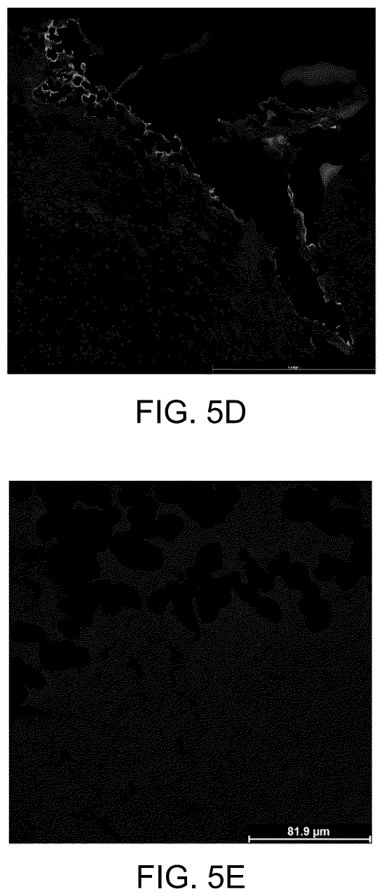

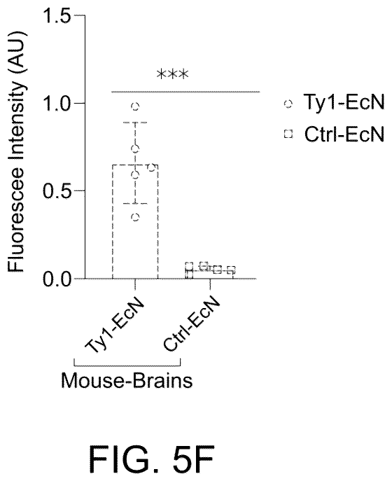

Ty1 expressing bacteria and control bacteria were orally administered in two different groups of mice, every day for 5 consequent days. Organs were subjected to OCT-embedded immunohistochemistry and stained with anti-Flag tag antibody and images were taken using Confocal microscopy. Lungs were harvested from Ty1 (FIG. 5A) and control bacteria (FIG. 5B). Images were quantified using ImagesJ Fiji (FIG. 5C). Brains were harvested from Ty1 (FIG. 5D) and control bacteria (FIG. 5E). They were quantified using ImageJ Fiji and plotted using Graph Pad Prism 8.0 v, significance was determined using unpaired t²-test, level of significance was set as “*” p<0.05; “**” p<0.01; “***” p<0.001, ns—not significant.

Following organ harvest, lungs and brains were subjected to immunofluorescence analysis using anti-flag tag antibodies. OMVs were observed in lungs of the animals orally immunized with Ty1 expressing bacteria (FIG. 5A), however no Ty1 were detected on the OMVs of control bacteria (FIG. 5B), which were significantly different as anticipated (FIG. 5C). Similarly, OMVs were also detected in brain of these animals (FIG. 5D) but were completely absent in control animals (FIG. 5E), which were significantly different (FIG. 5F). These data suggests that the nanobodies due to their nanoscale size can cross the blood-brain barrier.

### Example 6: OMVs Bearing Anti-Spike Nanobodies Inhibit Pseudo Viruses Expressing Spike Protein from Binding with ACE2 Receptor

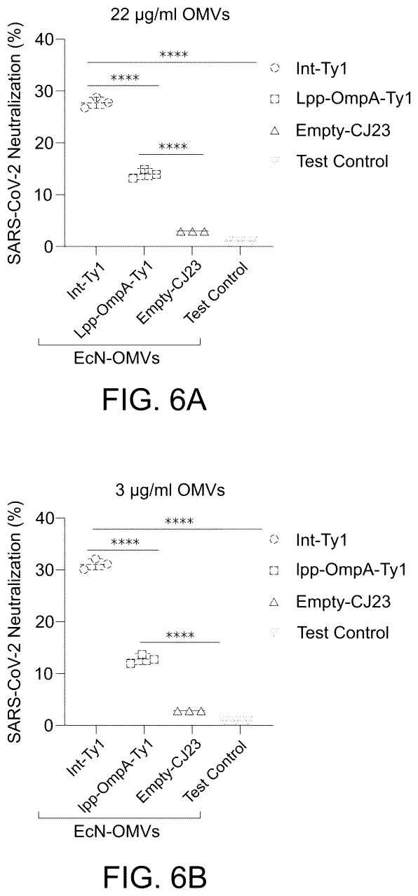

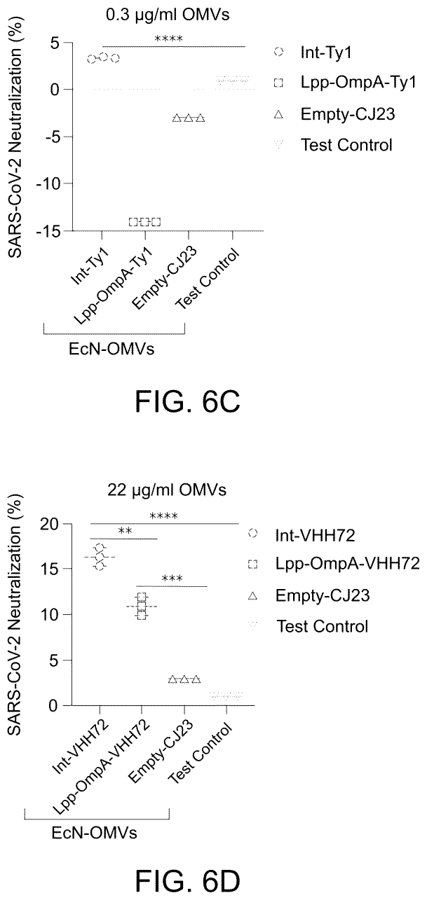

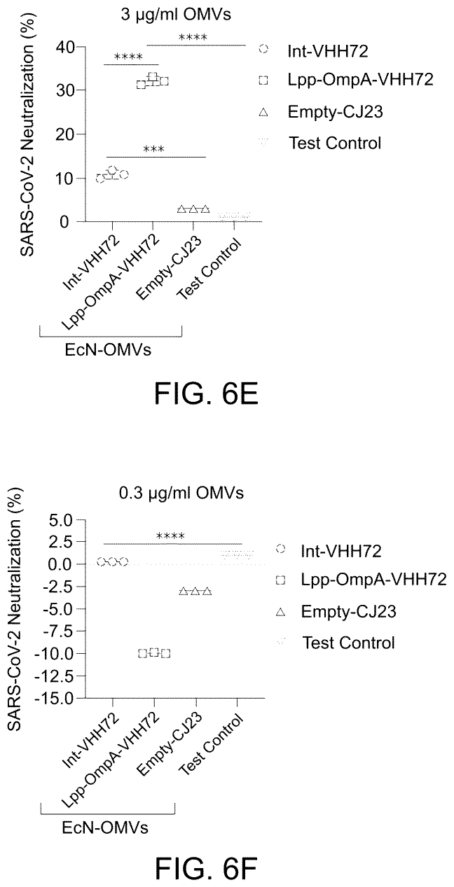

OMVs isolated from both control EcN and EcN surface expressing anti-Spike nanobodies-Ty1 and VHH72, were used in Pseudo virus-ACE2 neutralizing assay. Three different concentrations of OMVs were evaluated: 22 μg/mL, 3 μg/mL, and 0.3 μg/mL for the assay. At a concentration of 22 μg/mL, OMVs bearing Intimin-Ty1 nanobody showed the highest Pseudovirus-ACE2 receptor inhibition (˜30-32%), compared to OMV bearing Lpp-OmpA-Ty1 (15-18%) (FIG. 6A). Similarly, at 3 μg/mL OMV concentration, pInt-Ty1 (31-34%) showed significantly higher Pseudovirus-ACE2 receptor inhibition compared to Lpp-OmpA-Ty1 (15-18%) (FIG. 6B). However, at 0.3 μg/mL OMV concentration, Pseudovirus-ACE2 inhibition was only observed with pInt-Ty1 (3-4%) and not with Lpp-OmpA-Ty1 (FIG. 6C).

With the VHH72 constructs, pInt-VHH72 showed higher Pseudovirus-ACE2 receptor inhibition (15-16%) compared to pLpp-OmpA-VHH72 (10-11%), at an OMV concentration of 22 μg/mL, (FIG. 6D). At 3 μg/mL OMV concentration, pInt-VHH72 showed significantly higher Pseudovirus-ACE2 receptor inhibition (30-35%) compared to pLpp-OmpA-VHH72 (10%) (FIG. 6E). At 0.3 μg/mL OMV concentration, no significant Pseudovirus-ACE2 receptor inhibition was observed using either pInt-VHH72 or pLpp-OmpA-VHH72 (FIG. 6F). This data suggests that Intimin as an anchor protein is successfully able to display the nanobodies in an orientation conducive to neutralize the interaction of pseudo virus with ACE2 receptor, compared to pLpp-OmpA based anchors. In addition, Ty1 nanobodies were able to neutralize the interaction of pseudo virus with ACE2 receptor even at concentrations as low as 0.3 μg/mL, whereas with VHH72 nanobodies, the effectiveness drops significantly at concentrations<3 μg/mL. These data shows that the systematic development of a novel nanobody-based therapeutic platform and an OMV-based delivery system, which might help us combat emerging enigmatic diseases in the modern world.

### Example 7: Expression of Spike Protein on the Surface of EcN

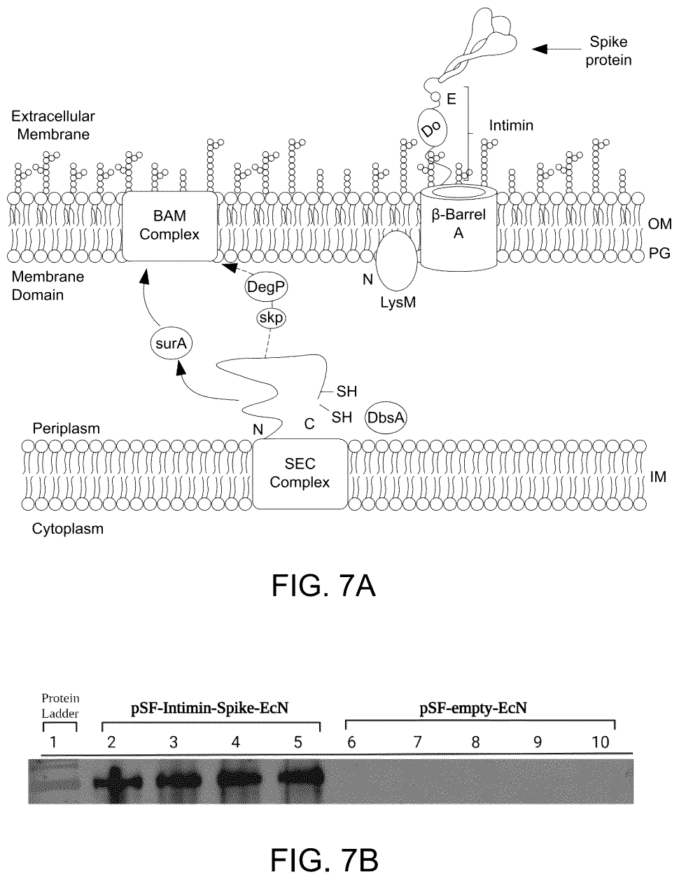

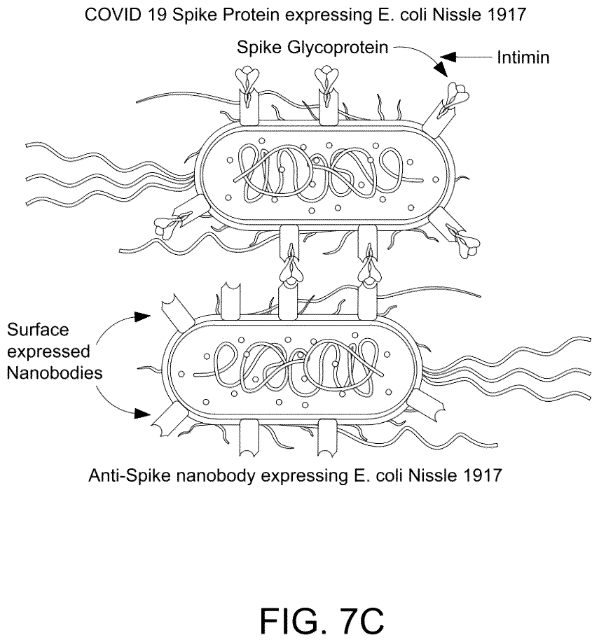

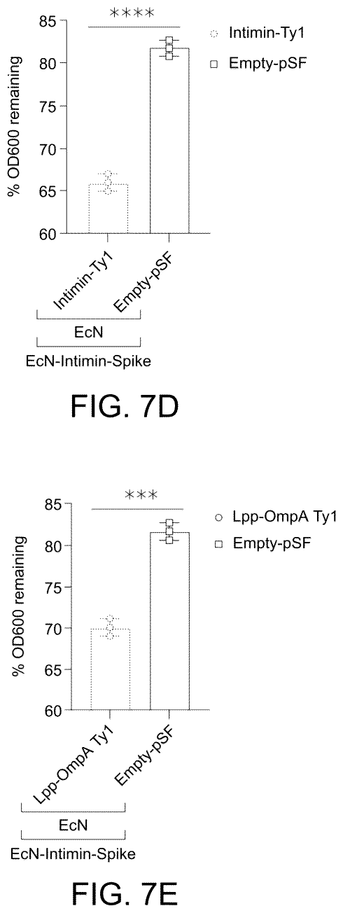

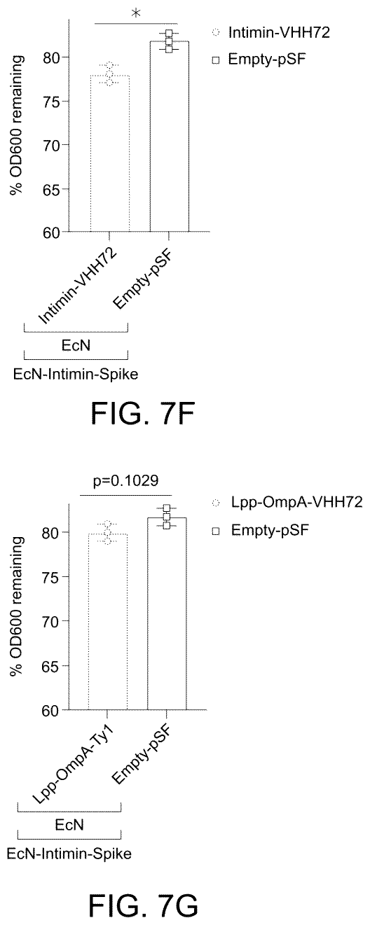

A new construct for the purpose of generating active immunity was developed by expressing spike protein on the surface of EcN. Intimin was used to anchor the SARS-COV-2 Spike protein on the bacterial cell surface (FIG. 7A and FIGS. 11A-11D). Confirmation of intimin-spike fusion protein expression was performed with SDS-PAGE Western blot analysis. Presence of ˜106.4 kDa band, corresponding to the size of Intimin-spike, and complete absence of any bands with EcN-expressing pSF-empty plasmid confirms the successful expression of spike protein on EcN (FIG. 7B). It was investigated whether the EcN-spike constructs would interact and bind to previously expressed cognate EcN-nanobody constructs, potentially leading to aggregation and precipitation of the complexes (FIG. 7C) (Glass and Riedel-Kruse, 2018; Prahlad et al., 2021). Following incubation of nanobody-bearing EcN with spike-bearing EcN, the bacteria readily aggregated, leading to a significant decrease in optical cell density (OD600) (Glass and Riedel-Kruse, 2018). pInt-Ty1, Lpp-OmpA-Ty1, and pInt-VHH72 nanobody expressing EcN showed significant aggregation with spike expressing EcN compared to WT-EcN in the following order from high to low: pInt-Ty1>Lpp-OmpA-Ty1>pInt-VHH72 (FIGS. 7D-7F). However, we did not see any significant difference in aggregation percent between Lpp-OmpA-VHH72 Nb and WT-EcN (FIG. 7G). These results indicate successful expression of functional surface displayed spike protein on EcN that is capable of binding to cognate proteins. In addition, these results further demonstrate that Intimin as an anchor protein outperforms Lpp-OmpA for both nanobody and spike protein expression on bacterial surface.

### Example 8: Oral Administration of EcN-Spike in Mice Induces an Anti-Spike Immune Response

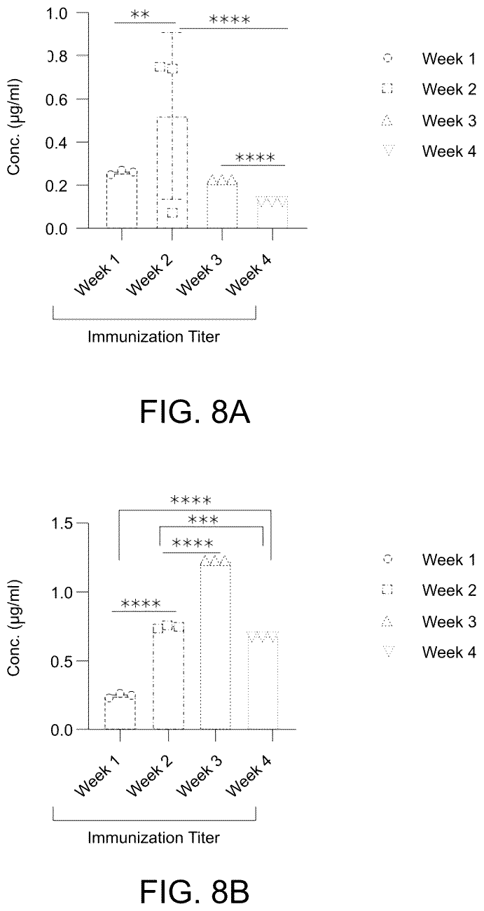

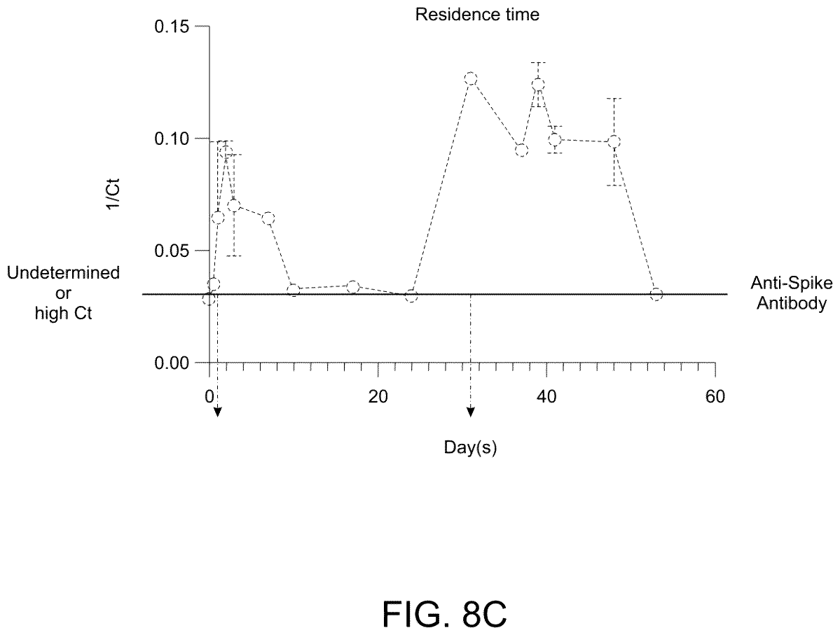

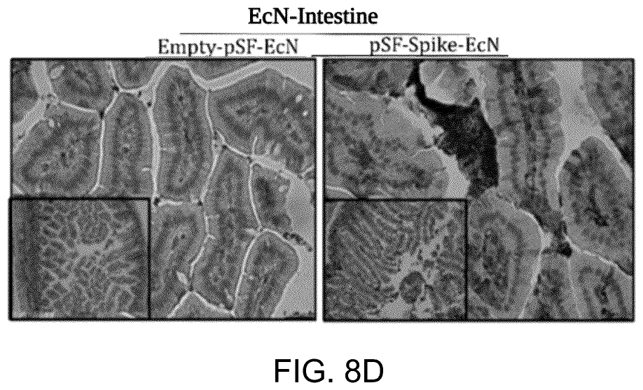

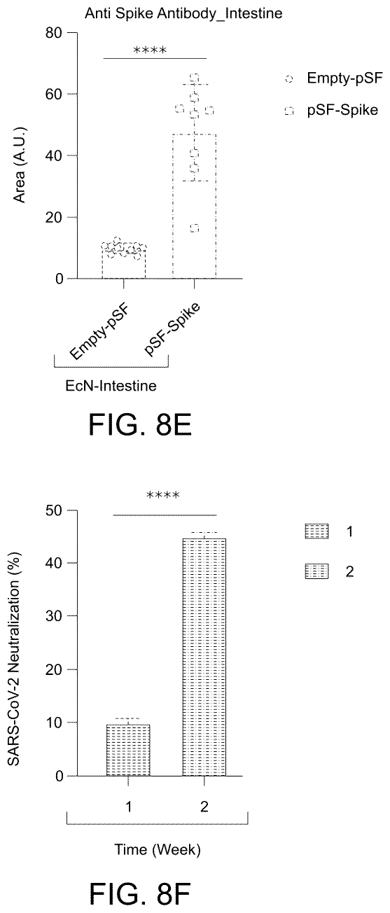

It was explored whether oral administration of EcN-spike would induce an immune response and generate anti-spike antibodies. EcN-spike co-expressing luciferase reporter was generated to facilitate imaging of the engineered bacteria using bioluminescence imaging (BLI). Single vs double dose of EcN-spike on antibody titre response was evaluated. For the single-dose regimen, EcN-spike-lux were administered orally once in naive C57BL6/J mice, and for the two-dose regimen EcN-spike-Lux was administered after 14 days. These mice were imaged every week for four weeks. Serum was also collected from the mice, including control mice which were administered with bacteria expressing empty plasmid. For one dose regimen, serum analysis revealed anti-spike antibody titre (˜0.2 μg/ml) after the first week of oral administration of EcN-spike. We obtained the highest levels in the second week (˜0.8 μg/ml), however, gradually decreasing after the third week and completely diminishing after the fourth week (FIG. 8A). Additionally, with the two-dose regimen of EcN-spike though, we observed a gradual increase in antibody titre levels, peaking at week three (˜1.25 μg/ml), and diminishing thereafter (FIG. 8B).

While BLI data analysis indicated there was a decrease in signal intensity suggesting decrease in total bacterial counts with the subsequent week of EcN-spike-lux administration, a more sensitive technique was sought to confirm these findings. Using qPCR analysis of mice fecal samples, it was observed that EcN has a typical residence time of ˜10-12 days in the gut after single oral administration (FIG. 8C), which likely explains the reason for diminished antibody titres after two weeks of oral administration. Although, BLI and qPCR analysis suggested the bacterial counts were attenuated, we sought to understand whether the engineered bacteria were completed eliminated from the system or whether some persisted. To confirm persistence of EcN-spike in the gut, an immunohistochemistry (IHC) analysis for Spike protein was performed after four weeks of administering the two-dose regimen. Three-fold higher levels of spike protein were observed in mice administered with EcN-spike compared to EcN alone (FIGS. 8D-8E). This suggests that significant levels of EcN-spike persist in the gut for the observed period, of four weeks. It also demonstrates the continuous and sustained expression of spike protein by the engineered construct and stability of the plasmid in the gut environment.

Next, it was investigated whether the antibody generated in response to oral administration of EcN-spike would inhibit spike protein and ACE2 receptor interaction. Serum samples were collected from mice administered with EcN-spike and EcN alone and tested using Pseudo virus neutralizing antibody assay. Serum collected after a week of oral administration of EcN-spike showed only ˜10% SARS-COV-2 neutralization by inhibiting the interaction between spike expressing Pseudoviruses and the ACE2 receptor on 293T-hACE2 cells (FIG. 8F). Serum collected from the second week of bacterial oral administration showed a higher, ˜44.65% inhibition of spike expressing Pseudo viruses and the ACE2 receptor. However, a significant decrease in neutralization was observed from the mice serum collected after the third week of oral administration of EcN-spike. Similarly, serum from the fourth week of administration did not show any significant difference in the neutralization between the control and the test serum samples. These results are in concordance with observed levels of antibody titres that peak at week two and suggest the antibodies that are generated indeed neutralize spike and ACE2 interaction.

### Example 9: Anti-Spike Antibodies Inhibit Pseudo Viruses from Interacting with ACE-2

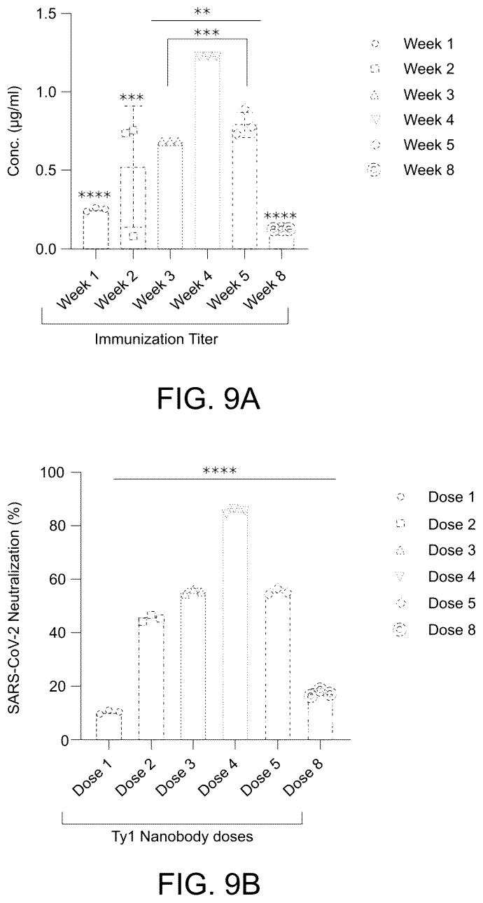

In a different approach, the oral administration of spike expressing bacteria was tested in mice gut for 8 weeks. Bacteria were administered every week for first 5 weeks and then 8th week. Resulting serum collected and quantified for antibody titer generation (FIG. 9A) and used in Pseudo virus neutralization assay, as previously. Antibody titer gradually increased with bacterial doses from dose (˜0.25 μg/ml) to (˜1.25 μg/ml) after dose 5 and gradually declining after 8th dose (˜0.12 μg/ml). Peak titer after 4th dose sand decline afterwards might be suggestive of saturation of antibody response. Finally, these antibodies were used for pseudo virus and ACE2 neutralization assay. A significantly higher correlation was observed between antibody titer and pseudo virus neutralization percentage. Neutralization percent gradually increased with doses of spike-EcN expressing bacteria in the mice gut. Pseudovirus-ACE2 neutralization of ˜10% was obtained after 1st dose, ˜45% after 2nd dose, ˜54% after 3rd dose and ˜85% after 4th dose and it gradually declined to ˜55% after 5th dose to ˜17% after 8th dose (FIG. 9B).

### Example 10: EcN-Spike Induces Mucosal Immunity

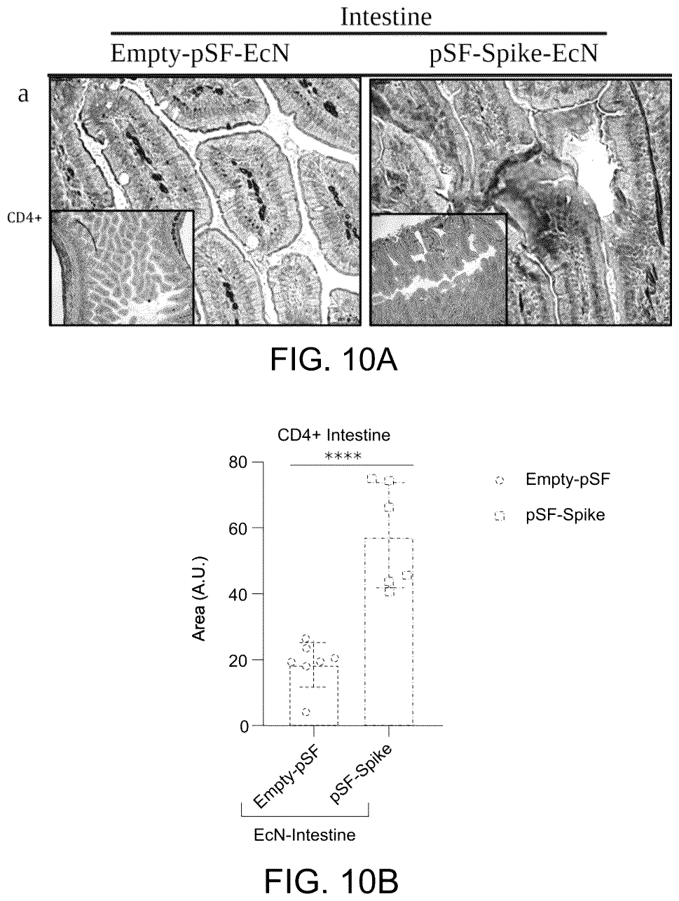

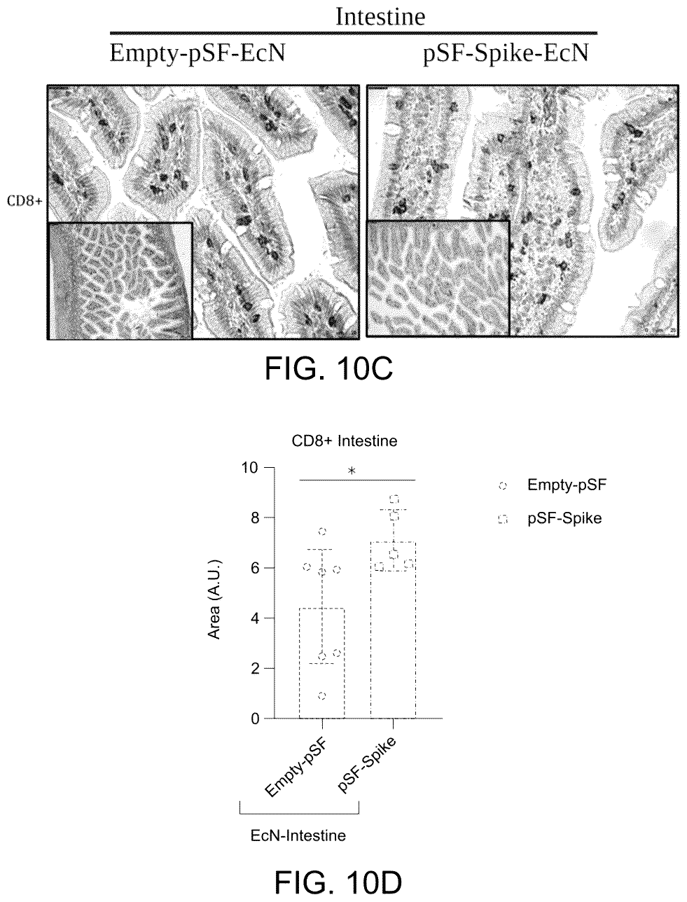

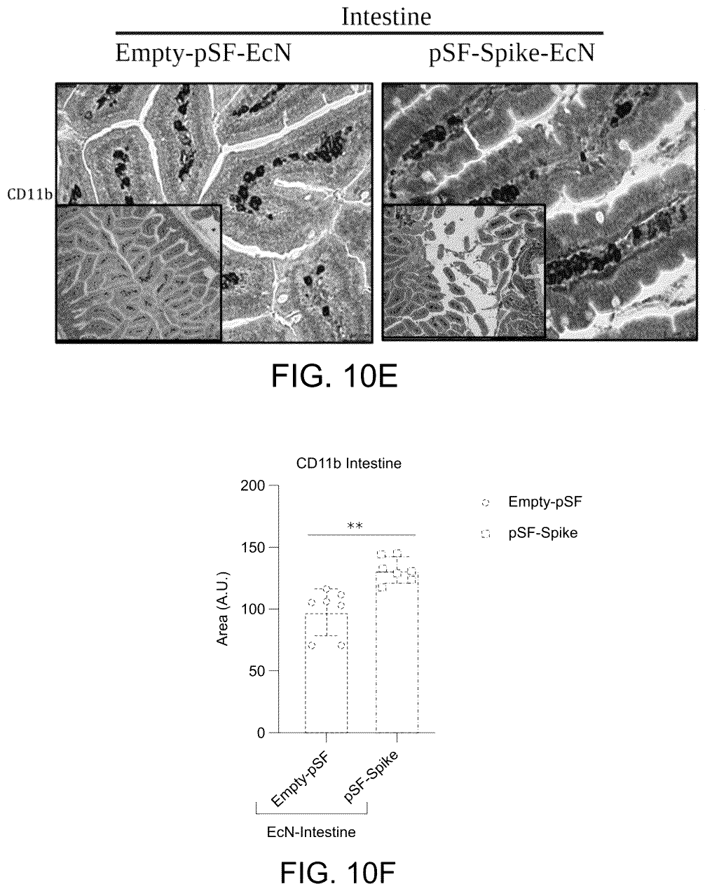

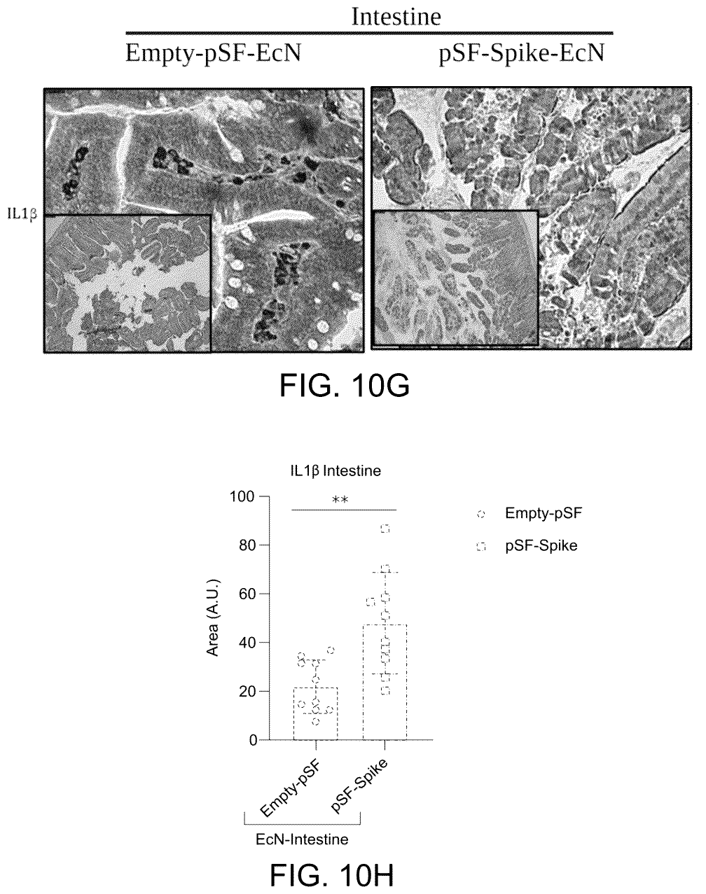

To assess whether EcN-Spike, in addition to generating systemic immunity, also induces mucosal immunity in the gut, the intestines were analysed four weeks after orally administering bacteria. IHC analysis was performed for the presence of lymphoid T cells and myeloid cells. Significantly higher levels of CD4+, CD8+ T-cells and activated monocytes (CD11b) were observed in mice orally administered with EcN-Spike compared to EcN alone as shown in FIGS. 10A-10F. Significantly higher levels of pro-inflammatory cytokine, IL1β were also observed in the intestines orally administered with Spike expressing bacteria than those that received control bacteria, as shown in FIGS. 10G and 10H. This data indicates that EcN-spike generates mucosal immunity in the gut through increased activation of T cells, monocytes and inducing release of pro-inflammatory cytokines.

### Bacterial Strains and Culture Growth Conditions

E. coli Nissle 1917 (EcN) was strain was used for the construction of all the bacterial strains bearing nanobodies (FIG. 12). All the strains were grown in LB broth or on LB agar plates, supplemented with appropriate antibiotic-Ampicillin (100 μg mL-1), Chloramphenicol (50 μg mL-1), or Kanamycin (100 μg mL-1). Prior to assaying protein expression with SDS-PAGE Western blot analysis, Fluorescence microscopy and Spike-ACE 2 inhibition assays, fresh media was inoculated with bacterial cultures from an overnight liquid culture. All the bacterial cultures were grown at 37° C. and 200 rpm.

Proteomics Analysis with Mass Spectrometry

Bacteria were grown as previously for proteomics analysis. In brief, 1% of overnight cultures were inoculated into 100 ml of fresh LB broth supplemented with the appropriate antibiotic and similarly grown at 37° C. and 200 rpm until OD600 reaches 0.9-1.0 (approx. 3.00-6.00 hr.). Cultures were spun at 4° C. for 15 min, 3500×g and cells were resuspended in 5-10 volumes of the Bacterial cell lysis buffer (Gold Bio), supplemented with DTT and EDTA (5 mM), and Lysozyme (40 mg/ml), DNase (800 U/ml) and RNase (24 U/ml). Following vertexing, and 5 min incubation on ice, suspensions were incubated at 37° C. for 60 min and lysates were centrifuged at 20,000×g, 4° C. for 30 min, and the clear lysate was collected and quantified using BCA assay (Thermo Scientific).

Protein samples were dried in a speed vac and resuspended in TEAB buffer according to standard in-solution digestion protocol. Samples were reduced with TCEP (tris-(2-carboxyethyl) phosphine) and alkylated with MMTS (methyl-methane-thiosulfonate). Samples were digested overnight at 37° C. and reactions were stopped by adding 10% formic acid. These samples were dried and resuspended in 0.1% formic acid. 5 μL (˜1 μg) of each sample was analyzed by NanoLC-MS/MS (Orbitrap Eclipse) and was searched against a combined database consisting of the E. coli Nissle 1917 database accessed from the Biocyc.org website and a database containing the nanobody sequences using Proteome discoverer ver 2.4 and the Sequest HT search algorithm using standard LFQ workflow (Thermo scientific).

### Tissue Culture

The metastatic triple negative murine breast cancer 4T1 cells (ATCC CRL-2539, passage number 5 to 15), CACO2 (passage number 5 to 15), and HEK293 (passage number 5 to 15), cell lines were cultured using RPMI media (Gibco #21875034) containing 10% Fetal Bovine Serum (Gibco #26140079) and 5% Penicillin-streptomycin (Gibco #15070063). 293T-hACE2 (Abnova Cat #KA6152) cell lines were cultured using DMEM media, supplemented with 10% FBS. Cells were maintained at 37° C. with 5% CO2 in air and sub-cultured 2 times a week, unless otherwise stated.

### Recombinant DNA Techniques

Plasmids generated during this study are listed in FIG. 12. High fidelity and diagnostic polymerase chain reactions (PCR) were performed using Phusion High Fidelity polymerase (New England Biolabs) and DreamTaq DNA Polymerase (Thermo Scientific), respectively. Geneblocks (GeneArtSynthesis) and custom primers (Integrated DNA technologies) used are listed in Table 2. Anti-COVID-Nanobody expressing genetic constructs with Intimin, and Lpp-OmpA surface display signals were designed, synthesized from GeneArtSynthesis® (Thermo Scientific) and Integrated DNA Technologies (IDT Inc.), and then incorporated into CJ23105 plasmid backbone using DNA 2.0 and Snap Gene® Viewer 4.1.8. These plasmids are listed in FIG. 12 and Table 3. All restriction digests and ligation reactions were carried out using NEB restriction enzymes and T4 DNA ligase (New England Biolabs). Following completion of ligation, reaction mixtures were chemically transformed into NEB DH5 α cells (New England Biolabs), as per the manufacturer's instructions.

### SDS-PAGE and Western Immunoblot Analysis

Following cell growth for expression assays, 1.0 OD unit of cultures samples were centrifuged at 16,000×g for 15 min to ensure separation of cells and supernatant. Supernatant fraction was prepared by using 10% v/v Trichloroacetic acid precipitation method followed by washing with ice-cold Acetone49. Both the precipitated supernatant and cell pellets were resuspended in 2×SDS loading buffer (10 ml Glycerol, 1 g SDS, 0.1 g Bromophenol Blue, 200 mM DTT to a volume of 50 mL in 100 mM Tris-HCL, pH 6.8) to a final volume of 50 μL and 200 μL, respectively. Both these cell fractions and supernatants were subjected to SDS-polyacrylamide gel electrophoresis (SDS-PAGE). Gels were Coomassie stained with Instant Blue (Expedeon™), Silver stained with Pierce™ Silver stain kit (Thermo Fisher Scientific) or prepared for Western immunoblotting by electro-transfer onto Nitrocellulose membrane (GE Healthcare Life Sciences). Blots were blocked using 3% w/v bovine serum albumin (BSA) in TBS buffer (24 g L-1 Tris Base, 88 g L-1 NaCl and 0.1% v/v Tween). Primary antibodies were diluted in 1:1000 using TBS-Tween. All blocking steps were carried at 4° C. overnight unless otherwise stated, while incubation steps were carried out at 2 hr unless otherwise stated. Western immunoblots were visualized using Pierce® ECL Western Blotting Substrates (Thermo Scientific) and ChemiDoc™ Imaging system (BioRad).

### COVID-19 Spike Protein-ACE2 Receptor Binding Inhibition Assay

EcN harboring 4-nanobodies expressing constructs, namely 1. pInt-Ty1 Nb, 2. pInt-VHH72 Nb, 3. pLpp-OmpA-Ty1 Nb and 4. pLpp-OmpA-VHH72 Nb along with wild-type E. coli Nissle 1917 as a control were grown in fresh LB broth as mentioned earlier. These cultures were then centrifuged at 4° C. for 10 min at 8400×g and cells were washed twice with PBS, centrifuged, and resuspended in 10 μl of PBS.

All the reagents were brought to RT (18-25° C.) before use and reactions were run in triplicates. Test samples for Nanobody inhibiting Spike protein-Receptor binding domain (RBD) and ACE-2 receptor interaction were prepared as below. Briefly, 2 ml of Nanobody expressing cultures along with wild-type WT-EcN were normalized to OD600=1.0 Unit. Cultures were centrifuged twice at 8400 rpm for 15 min and resuspended in 1 ml of PBS. Stock solution of test reagent was prepared by mixing 75 μL Bacterial cell suspension in PBS, 7.5 μL, 50×ACE2 protein concentrate and 292.5 μL 1× Assay diluent, for preparing sample enough for triplicates. Following dilutions were prepared by using 37.5 μL previous sample and adding into 337.5 μL, 1×ACE2 protein working solution, while positive control was prepared by using only 375 μL, 1×ACE2 protein working solution, for triplicates at 100 μL per well.

Spike-RBD coated 96-well plate was labelled and 100 μL each of Test reagent sample was added into appropriate well, covered with plate sealing film and incubated overnight at 4° C. with gentle shaking. Following day, the solution was discarded, washed 4× times with 1× wash solution and added with 300 μL of 1× wash buffer for 5 min and liquid was completely removed. After the last wash, residual 1× wash buffer was removed with decanting or aspirating and added with 100 μL of 1× Detection Antibody and incubated 1 hr at RT. After incubation, solution was discarded and wash steps were repeated 3× times, as previously. Plate was then added with 1× Anti Goat HR-conjugated IgG antibody to each well and incubated for 1 hr at RT. Solution was discarded and wash steps were repeated as previously for 4× times. Each well was then added with 100 μL of TMB One-Step substrate Reagent and incubated 30 min at RT in dark. It was added with 50 μL of Stop Solution and absorbance was read at 450 nm, using EnVision 2102 Multilabel Reader (Perkin Elmer).

### Development of an In-House Assay for Surface Nanobody Detection

EcN were freshly transformed with nanobody expressing plasmids and grown along with WT E. coli Nissle 1917 (EcN) as a control. All cultures were grown identically in 10 ml LB medium using an overnight culture and supplemented with appropriate antibiotic, for ˜3.00 hr or until OD600 reached 0.9-1.0. All the centrifugation steps were carried out at 3500×g for 15 min, at RT, unless otherwise stated. OD600, 1 Unit samples from each culture were centrifuged and pellets were resuspended in 1 ml of Phosphate Buffered Saline (PBS). It was centrifuged and pellets were re-suspended in 200 μl of PBS and added with 1 μg of Spike Protein (carrier-free Recombinant SARS-CoV-2 S Protein (S1+S2) (Bioline #793706)) and incubated in the dark for an hour at RT. Following centrifugation, it was resuspended in 1 ml of PBS and centrifuged again, and pellet were resuspended into 200 μl of PBS. It was then added with 1 μg of Purified anti-SARS-COV-2 Protein S2 antibody (Bioline #943202), that specifically binds to S2 fragment of spike protein. This whole reaction was incubated in the dark for 30 min. It was centrifuged and resuspended into 1 ml of PBS as previously and centrifuged. Following this, pellet was re-suspended into 200 μl of PBS and added with secondary antibody (Alexa Fluor® 647 anti-mouse IgG2b, Bioline #406715). Following 30 min of incubation in dark, the reaction tubes were centrifuged twice, and pellet were resuspended into 200 μl of PBS. It was placed into a transparent 96-well (Corning®) plate and read for the fluorescence using a fluorescent microscope (Leica Microsystems).

### Competitive Exclusion Assay to Evaluate Nanobody Catalyzed Inhibition of Spike-RBD-ACE2 Receptor Binding Using CaCo2 and 4T1 Cell Lines

CaCO2, HEK293FT, and 4T1 cell lines were used for the nanobody detection assay. Cell lines were grown in respective media (ATCC) using a 8-chamber slide (Ibidi), until confluency by incubating at 37° C. with 5% CO2. The cell media were removed, and cells were washed 3× times with PBS. Cells were fixed using 4% formaldehyde (Fisher Brand) by incubating for 10 min at RT. The formaldehyde was drained, and cells were washed 3× times to remove the residual formaldehyde. It was followed by permeabilization. Cells were permeabilized with permeabilization buffer (0.1% Triton x 100 in PBS) and incubated for 10 min at RT, followed by 3× washes with PBS. Cells were then blocked using blocking buffer (3% BSA in PBS with 0.1% Triton X100) for 30 min at RT. Following buffer removal, cells were added with primary antibody and/or spike protein prepared in 0.1% Triton X 100, with 30 mg/mL of BSA (Filter sterilized). Primary antibody (Anti-ACE2 (E-11): sc-390851 (Santa Cruz Biotechnology Inc.)) was added for 1-2 hr at RT followed with 3× washes with wash Buffer (0.1% Triton X 100 in PBS) (15 min washing with 5 min in between). It was followed with secondary antibody (Alexa Fluor® 647 anti-mouse IgG2b, Bioline #406715) prepared in PBS (0.1% Triton x100 with 30 mg/ml BSA).

When Spike protein was utilized, following the blocking step, and/or Anti-ACE2 washing step, cells were added with 1 μg of Spike Protein (carrier-free Recombinant SARS-COV-2 S Protein (S1+S2) (Bioline #793706)) and incubated in dark for an hour at RT. Following 3× washes with washing buffer, 1 μg of Purified anti-SARS-COV-2 Protein S2 antibody (Bioline #943202) was added, that specifically binds to S2 fragment of Spike Protein. The whole reaction was incubated in dark for 30 min. Following 3× washes with washing buffer, secondary antibody (Alexa Fluor® 647 anti-mouse IgG2b, Bioline #406715) was added. After 30 min of incubation in dark, cells were washed 3× times with washing buffer and were visualized for fluorescence, using a fluorescent microscope (Leica Microsystems) at The Live Microscopy Imaging Core, The University of Cincinnati.

### Isolation and Characterization of Outer Membrane Vesicles (OMV)

For the isolation of bacterial outer membrane vesicles (OMV) a sequential differential centrifugation protocol was developed. In brief, anti-COVID nanobody bearing EcN along with WT-EcN were grown overnight at 37° C. and 200 rpm. 1.0% of these cultures were inoculated in 1.0 L of fresh LB Broth, supplemented with appropriate antibiotic. Cultures were grown until OD600 of ˜1.5 Unit. Culture was cleared of bacteria by centrifugation (8000×g, 4° C., 15 minutes) followed by concentrating the supernatant using Pierce Protein Concentrators (30 kDa, ThermoFisher #88531). 70 ml of the concentrated supernatant was then ultracentrifuged at 91,000×g for 4.0 hr. The pellet obtained was resuspended in PBS, pH 7.4, followed by washing with Amicon Ultra-0.5 Centrifugal Filter Unit (Millipore #UFC500308) filters to completely remove the residual media. The resulting dispersion was sterile filtered through 0.22 μm PVDF syringe filters (Cole-Parmer #UX-06060-62). OMVs were then characterized for size and size distribution using Dynamic Light Scattering (Zetasizer Nano ZS, Malvern Instruments). Protein concentration and OMVs were quantified with a Pierce™ BCA Protein Assay kit (Thermo Scientific), using BSA standards. Protein standards and OMVs were aliquoted and stored at −20° C.

### Immunofluorescence Microscopy

Immunofluorescence microscopy was used to investigate the presentation of nanobodies on the cell surface. A 200 μL of nanobody expressing pInt-VHH72 Nb, pInt-Ty1 Nb, pLpp-OmpA-VHH72 Nb, and pLpp-OmpA-Ty1 Nb recombinant bacterial cells were harvested and centrifuged at 3500×g for 5 min and washed 3× times with PBS (pH 7.4), supplemented with 3% BSA and incubated with recombinant Spike Protein (carrier-free Recombinant SARS-COV-2 S Protein (S1+S2) (Bioline #793706)). Following three washes, Nanobody-Spike protein complex was added with anti-SARS-COV-2 Protein S2 antibody (Bioline #943202) and incubated in dark for 1 hr at RT. After washing 3× times with PBS, nanobody-spike-antibody complex was incubated 1.5 hr at RT with anti-mouse IgG2 antibody conjugated with Alexa Fluor® 647 (Bioline #406715). For microscopic observations, cells were washed 5× times with PBS solution to remove unbound Alexa Fluor® 647 antibody, then mounted on microscopic slide or in a 96-well plate and observed by fluorescence microscopy.

### Antibody Titer Generation in Response to Oral Administration of Spike Expressing Bacteria in the Mouse Gut

All the animal experiments were conducted by following a protocol approved by the University of Cincinnati Biosafety, Radiation safety, and Animal Care and Use Committees (20-05-16-01). Spike protein-expressing bacteria (˜1×107) were orally administered in 6-9-week-old male C57BL/6 mice, every week for four consecutive weeks and their blood was withdrawn. Blood was spun at 2000×g for 30 min at 4° C. and serum was collected in an Eppendorf tube. Mouse Anti-SARS-CoV-2 Antibody IgG Titer Serologic Assay Kit (Spike timer) was used to determine the antibody titer in the serum sample, according to the manufacturer's instructions (Acro Biosystems). In brief, 100 μl of diluted serum sample, along with blank, positive, and negative control were added into a pre-coated SARS-COV-2 Spike protein microplate and incubated at 37° C. for 1 hr. Following three washes with 1× washing buffer HRP-Goat anti-Mouse IgG was added and incubated at 37° C. for 1 hr in the dark. Following 3× washes, plate was added with 100 μl substrate solution and incubated at 37° C. for 20 min, followed by termination of the reaction by adding 50 μl of Stop solution. Absorbance was read at 450 nm using UV/Vis's microplate spectrophotometer.

### COVID-19 Pseudo Virus Neutralizing Antibody Assay (Luciferase)

COVID-19 Pseudo virus Neutralizing Antibody Assay (Luciferase) was performed as per the manufacturer's instructions (Abnova). In brief, prior to assay 293T-hACE2 cells (˜1×105 cells/well) were grown in a 24 well plate and grown at 37° C. for 4.00 hr. Following incubation, media was removed, and cells were washed twice with PBS. Cells were added with Cell Dissociation media (HIMEDIA) and incubated for 5 min at 37° C. and added with a complete medium to neutralize the reaction and centrifuged at 300×g for 5 min. Pellet was resuspended in a complete medium to get 2×105 cells/mL. Meanwhile, 50 μl diluted sample (OMV, Serum, antibody, or bacteria) and 10 μl pseudovirus expressing luciferase were mixed and incubated at RT for 30 min prior to adding to the 293T-hACE 2 cells in a 24 well plate. Plates were incubated at 37° C. for 48 hr. Following incubation, medium was removed, and cells were gently washed with 200 μl PBS. It was added with 100 μl of Luciferase Cell Lysis Reagent and cells were scrapped off the plate, vortexed for 10-15 sec and centrifuged at 12,000×g for 30 second and restored on ice instantly. 10 μl of cell lysate and 50 μl of Luciferase Assay Reagent were added in a 96-well plate and luminescence was immediately read using an En Vision 2102 Multilabel Reader (Perkin Elmer) to detect the Luciferase expression.

### Bioluminescence Imaging

Bioluminescence images were acquired using the Perkin Elmers IVIS Spectrum In Vivo Imaging system (2 min exposure) for the quantification of Radiance (Photons/Sec/cm2) of the bioluminescent signals from the regions of interest.

### Immunohistochemistry

Small and Large Intestines were isolated from the mice gut and fixed with 10% v/v formalin for 24 hr. It was replaced with storage solution 70% v/v ethanol. Immunohistochemistry slides were prepared and developed by the Pathology Research Core at the Cincinnati Children's Hospital Medical Center via the paraffin processing. These slides were imaged under ×100 and ×400 magnification using Leica DMi8 Widefield fluorescence/Brightfield Microscope. The images were quantified using ImageJ as previously reported elsewhere.

### Statistical Analyses

All statistical analyses were performed using the GraphPad Prism 8.0.v. software. Ordinary one-way ANOVA was used to compare means between distinct groups, with at least 3 or more biological replicates. When two groups were compared two-tailed t-tests were used. Asterisks in the graphs represents that the mean differences were statistically significant (p<0.05). The level of significance was set as “*” p<0.05; “**” p<0.01; “***” p<0.001, ns—not significant.

### Abbreviations

Nb—Nanobody; EP—Engineered probiotic bacterium; OMV—Outer Membrane Vesicles

### Tables

TABLE 1
Antibodies used

| Antibody | Dilution | Application |
|---|---|---|
| DYKDDDDK Tag | 1:10,000 | Western blot |
| Monoclonal Antibody | | |
| (FGGR) HRP Invitrogen ™ | | |
| (Thermo Fisher) | | |
| Alexa Fluor ®647 anti-mouse | | Immunohistochemistry |
| IgG2b (#406715, | | |
| BioLegend ®) | | |
| PE anti-mouse IgG2b, | | Immunohistochemistry |
| (#406707, BioLegend ®) | | |
| Purified anti-SARS-CoV-2 S | | Immunohistochemistry |
| protein S2, (#943202, | | |
| BioLegend ®) | | |
| Recombinant SARS-CoV-2 S | | Immunohistochemistry |
| protein S1 + S2 (Carrier free), | | |
| #793706, BioLegend ® | | |
| Anti-ACE2 (E-11): | 1:100 | Immunohistochemistry |
| sc-390851 (Santa Cruz | | |
| Biotechnology Inc.) | | |
| SARS-CoV-2 Spike Protein | 1:3000 | Fluorescence |
| (RBD), mFc Tag (#100684, | | Microscopy |
| BPS Biosciences) | | |
| ACE, His-Tag (#11003, BPS | 1:1000 | Fluorescence |
| Biosciences) | | Microscopy, |
| | | Bioassay |
| Anti-His-HRP (BPS | 1:5000 | Fluorescence |
| Biosciences) | | Microscopy, |
| | | Bioassay |
| Antibody for the detection of | 1:10000-1:25000 | FLISA, IF |
| FLAG ™ conjugated proteins | | Microscopy, |
| (MOUSE) Monoclonal | | Western blot |
| antibody DyLight ™ 800 | | |
| conjugated #200-345-383 | | |
| (Rockland) | | |
| β-actin antibody -MA5- | 1:1000 | Western blotting |
| 15739-HRP (Invitrogen) | | |
| Detection Antibody Cocktail, | | Cytokine Array |
| Mouse Cytokine Array Panel | | |
| A | | |
| Streptavidin-HRP | 1:2000 | Cytokine Array |

TABLE 2
Gene blocks

| Plasmid | Geneblock/features | Antibiotic |
|---|---|---|
| pKM-RQ | Intimin-Ty1-TEV-strep/ | Km^{R} |
| | flag tag | |
| pKA-RQ | Lpp-OmpA-VHH72-TEV- | Amp^{R} |
| | strep/flag tag | |

TABLE 3
Plasmids generated

| Plasmid Constructs | Features/elements | Antibiotic resistance |
|---|---|---|
| PInt-WT-EcN | Empty plasmid cJ23105 | Cm^{R} |
| pInt-Ty1 | Intimin-Ty1 | Cm^{R} |
| pInt-VHH72 | Intimin-VHH72 | Cm^{R} |
| pLpp-OmpA-Ty1 | Lpp-OmpA-Ty1 | Cm^{R} |
| pLpp-OmpA-VHH72 | Lpp-OmpA-VHH72 | Cm^{R} |
| pInt-Spike-RBD | Intimin-Spike-RBD | Km^{R} |
| pLpp-OmpA-Spike-RBD | Lpp-OmpA-Spike -RBD | Km^{R} |

TABLE 4
Primers used

| | | Tm |
|---|---|---|
| Primer | Primer Sequence (5′-3′) | (° C.) |
| CJ23105 Seq | TTTACGGCTAGCTCAGTCCTAGGTA | 58.5 |
| 002F (pInt- | ACTAAGCTTGAAAATCTGTATTTTC | 68.5 |
| VHH72) | AGGGCACCCGTCAGGTTCAGCTGC | |
| 002R (pInt- | CTTACTCGAGTGGTACCTTAACGGC | 61.5 |
| VHH72) | TTTTTTCAAAC | |
| 003F (pLpp- | ATCHAATTCAAAAGATCTTTTAAGA | 59.7 |
| OmpA-Ty1) | AGGAGATATACATATGAAAG | |
| 003R (pLpp- | AGTAAGCTTGCGGGTTGCAATTTCC | 66.0 |
| OmpA-Ty1) | GGTGTAATTGC | |
| J23105 | GACGTCTTTACGGCTAGC | 63 |
| Neae_F | | |
| J23105_Seq | TTTACGGCTAGCTCAGTCC | 64 |

TABLE 5
Confirmation of pInt-Ty1 protein expression with Mass Spectrometry Analysis

| | Sequest | | | | Mol. |
|---|---|---|---|---|---|
| | HT | Abundance | | Calc. | WT |
| Nanobody | Score | ratio | Unique peptides | pI | (kDa) |
| Intimin- | 354.94 | 0.768 | [1]LIYEQYYGDNVALFNSDK | 8.38 | 89.3 |
| Ty1 Nb | | | [2]LPFEYSALPLLGSAPLVAAGGV | | |
| | | | AGHTNK | | |
| | | | [3]LQSNPGAATVGVNYTPIPLVTM | | |
| | | | GIDYR | | |
| | | | [4]LLTHDSYQNR | | |
| | | | [5]HLYSSESEMMK | | |
| | | | [6]KNGVAQANVPVSFNIVSGTATL | | |
| | | | GANSAK | | |
| | | | [7]KLPFEYSALPLLGSAPLVAAGG | | |
| | | | VAGHTNK | | |
| | | | [8]HGTGNENDLLYSMQFR | | |
| | | | [9]SQDINLSTIWSLNK | | |
| | | | [10]SQGGQIQHSGSQSAQDYQAILP | | |
| | | | AYVQGGSNIYK | | |
| | | | [11]QVQLVETGGGLVQPGGSLR | | |
| | | | [12]SWSQQIEPQYVNELR | | |
| | | | [13]ISPNSGNIGYTDSVK | | |
| | | | [14]NGVAQANVPVSFNIVSGTATL | | |
| | | | GANSAK | | |
| | | | [15]ALNYAAQQAASLGSQLQSR | | |
| | | | [16]ADNADTITYTATVK | | |
| | | | [17]FFLPANMLGYNVFIDQDFSGD | | |
| | | | N | | |
| | | | [18]AAPGQQIILPLK | | |
| | | | [19]DDDDKWSHPQFEK | | |
| | | | [20]IVWDDSALR | | |
| | | | [21]MLAFGQVGAR | | |
| | | | [22]MITHGCYTR | | |
| | | | [23]MSGWHESYNK | | |
| | | | [24]TGETVADLSK | | |
| | | | [25]SSTPGQVVVSAK | | |
| | | | [26]AAPGQQIILPLKK | | |
| | | | [27]NNNIILEYK | | |
| | | | [28]HLYSSESEMMK | | |
| | | | [29]LENLYFQGTR | | |
| | | | [30]FTANLGAGQR | | |
| | | | [31]LGIGGEYWR | | |
| | | | [32]WSHPQFEK | | |
| | | | [33]MSPDVTK | | |
| | | | [34]MSPDVTK | | |
| | | | [35]YDLVQR | | |
| | | | [36]GPEWVSR | | |
| | | | [37]SNMTDDK | | |

TABLE 6
Confirmation of pLpp-OmpA-Ty1 protein expression with Mass Spectrometry

| | | | | | Mol. |
|---|---|---|---|---|---|
| | Sequest | Abundance | | Calc. | WT |
| Nanobody | HT Score | ratio | Unique peptides | pI | (kDa) |
| Lpp-OmpA- | 95.92 | 0.992 | [1]LGYPITDDLDIYTR | 8.18 | 32.6 |
| Ty1 Nb | | | [2]QVQLVETGGGLVQPGGSLR | | |
| | | | [3]ISPNSGNIGYTDSVK | | |
| | | | [4]NHDTGVSPVFAGGVEYAITPEIATR | | |
| | | | [5]DDDDKWSHPQFEK | | |
| | | | [6]LENLYFQGTR | | |
| | | | [7]LGGMVWR | | |
| | | | [8]AQGVQLTAK | | |
| | | | [9]WSHPQFEK | | |
| | | | [10]GPEWVSR | | |
| | | | 11]KLENLYFQGTR | | |

The embodiments of the present invention recited herein are intended to be merely exemplary and those skilled in the art will be able to make numerous variations and modifications to it without departing from the spirit of the present invention. Notwithstanding the above, certain variations and modifications, while producing less than optimal results, may still produce satisfactory results. All such variations and modifications are intended to be within the scope of the present invention as defined by the claims appended hereto

SEQUENCES

Intimin-Ty1 nanobody sequence
MITHGCYTRTRHKHKLKKTLIMLSAGLLGLFFYVNQNSFANGENYFKLGSDSKLL
TIIDSYQNRLFYTLKTGETVADLSKSQDINLSTIWSLNKHLYSSESEMMKAAPGQQIILPLK
KLPFEYSALPLLGSAPLVAAGGVAGHTNKLTKMSPDVTKSNMTDDKALNYAAQQAASL
GSQLQSRSLNGDYAKDTALGIAGNQASSQLQAWLQHYGTAEVNLQSGNNFDGSSLDFLL
PFYDSEKMLAFGQVGARYIDSRFTANLGAGQRFFLPANMLGYNVFIDQDFSGDNTRLGIG
GEYWRDYFKSSVNGYFRMSGWHESYNKKDYDERPANGFDIRFNGYLPSYPALGAKLIYE
QYYGDNVALFNSDKLQSNPGAA1VGVNYTPIPLVTMGIDYRHGTGNENDLLYSMQFRYQ
FDKSWSQQIEPQYVNELRfLSGSRYDLVQRNNNILEEYKKQDILSNIPPHDING1EHSTQKIQ
LIVKSKYGLDRIVWDDSALRSQGGQIQHSGSQSAQDYQAILPAYVQGGSNIYKVTARAYD
RNGNSSNNVQLTIIVLSNGQVVDQVGVTDFTADKTSAKADNADTITYTA1VKKNGVAQA
NVPVSFNIVSGTATLGANSAKIDANGKATVTLKSSTPGQVVVSAKTAEMTSALNASAVIF
FDGAPGKLENLYFQGTRQVQLVETGGGLVQPGGSLRLSCAASGFTFSSVYMNWVRQAPG
KGPEWVSRISPNSGNIGYIDSVKGRFTISRDNAQNTLYLQMNNLKPEDTALYYCAIGLNLS
SSSVRGQGTQVTVSSGSENLYFQGDYKDDDDKWSHPQFEKSR* [SEQ ID NO: 1]
pInt-VHH72 sequence
MITHGCYTRTRHKHKLKKTLIMLSAGLLGLFFYVNQNSFANGENYFKLGSDSKLL
TIIDSYQNRLFYTLKTGETVADLSKSQDINLSTIWSLNKHLYSSESEMMKAAPGQQIILPLK
KLPFEYSALPLLGSAPLVAAGGVAGHTNKLTKMSPDVTKSNMTDDKALNYAAQQAASL
GSQLQSRSLNGDYAKDTALGIAGNQASSQLQAWLQHYGTAEVNLQSGNNFDGSSLDFLL
PFYDSEKMLAFGQVGARYIDSRFTANLGAGQRFFLPANMLGYNVFIDQDFSGDNTRLGIG
GEYWRDYFKSSVNGYFRMSGWHESYNKKDYDERPANGFDIRFNGYLPSYPALGAKLIYE
QYYGDNVALFNSDKLQSNPGAA1VGVNYTPIPLVTMGIDYRHGTGNENDLLYSMQFRYQ
FDKSWSQQIEPQYVNELRfLSGSRYDLVQRNNNILEEYKKQDILSNIPPHDINGIEHSTQKIQ
LIVKSKYGLDRIVWDDSALRSQGGQIQHSGSQSAQDYQAILPAYVQGGSNIYKVTARAYD
RNGNSSNNVQLTIIVLSNGQVVDQVGVTDFTADKTSAKADNADTITYTA1VKKNGVAQA
NVPVSFNIVSGTATLGANSAKIDANGKATVTLKSSTPGQVVVSAKTAEMTSALNASAVIF
FDGAPGKLENLYFQGTRQVQLQESGGGLVQAGGSLRLSCAASGRTFSEYAMGWFRQAP
GKEREFVATISWSGGSTYYTDSVKGRFTISRDNAKNTVYLQMNSLKPDDTAVYYCAAA
GLGTVVSEWDYDYDYWGQGTQVTVSSGSGSENLYFQGDYKDDDDKWSHPQFEKSR*
[SEQ ID NO: 2]
Lpp-OmpA-Ty1 nanobody
MKATKLVLGAVILGSTLLAGCSSNAKIDQGINNNGPTHENQLGAGAFGGYQVNPY
VGFEMGYDWLGRMPYKGSVENGAYKAQGVOLTAKLGYPITDDLDIYTRLGGMVWRAD
TKSNVYGKNHDTGVSPVFAGGVEYAITPEIATRKLENLYFQGTRQVQLVETGGGLVQPGG
SLRLSCAASGFTFSSVYMNWVRQAPGKGPEWVSRISPNSGNIGYTDSVKGRFTISRDNAK
NTLYLQMNNKPEDTALYYCAIGLNLSSSSVRGQGTQVTVSSGSENLYFQGDYKDDDDKW
SHPQFEKSR* [SEQ ID NO: 3]
pLpp-OmpA-VHH72 sequence
MKATKLVLGAVILGSTLLAGCSSNAKIDQGINNNGPTHENQLGAGAFGGYQVNPY
VGFEMGYDWLGRMPYKGSVENGAYKAQGVQLTAKLGYPITDDLDIYTRLGGMVWRAD
TKSNVYGKNHDTGVSPVFAGGVEYAITPEIATRKLENLYFQGTRQVQLQESGGGLVQAG
GSLRLSCAASGRTFSEYAMGWFRQAPGKEREFVATISWSGGSTYYTDSVKGRFTISRDN
AKNTVYLQMNSLKPDDTAVYYCAAAGLGTVVSEWDYDYDYWGQGTQVTVSSGSGSE
NLYFQGDYKDDDDKWSHPQFEKSR* [SEQ ID NO: 4]
SARS-COV-2 Spike protein Spike protein
MITHGCYTRTRHKHKLKKTLIMLSAGLLGLFFYVNQNSFANGENYFKLGSDSKLL
TIIDSYQNRLFYTLKTGETVADLSKSQDINLSTIWSLNKHLYSSESEMMKAAPGQQIILPLK
KLPFEYSALPLLGSAPLVAAGGVAGHTNKLTKMSPDVTKSNMTDDKALNYAAQQAASL
GSQLQSRSLNGDYAKDTALGIAGNQASSQLQAWLQHYGTAEVNLQSGNNFDGSSLDFLL
PFYDSEKMLAFGQVGARYIDSRFTANLGAGQRFFLPANMLGYNVFIDQDFSGDNTRLGIG
GEYWRDYFKSSVNGYFRMSGWHESYNKKDYDERPANGFDIRFNGYLPSYPALGAKLIYE
QYYGDNVALFNSDKLQSNPGAA1VGVNYTPIPLVTMGIDYRHGTGNENDLLYSMQFRYQ
FDKSWSQQIEPQYVNELRfLSGSRYDLVQRNNNILEEYKKQDILSNIPPHDING1EHSTQKIQ
LIVKSKYGLDRIVWDDSALRSQGGQIQHSGSQSAQDYQAILPAYVQGGSNIYKVTARAYD
RNGNSSNNVQLTIIVLSNGQVVDQVGVTDFTADKTSAKADNADTITYTA1VKKNGVAQA
NVPVSFNIVSGTATLGANSAKIDANGKATVTLKSSTPGQVVVSAKTAEMTSALNASAVIF
FDGAPGKLENLYFQGTRMRVQPTESIVRFPNITNLCPFGEVENATRFASVYAWNRKRISNC
VADVSVLYNSASFSTFKCYGVSPTKLNDLCFTNVYADSFVIRGDEVRQIAPGQTGKIADY
NYKLPDDFTGCVIAWNSNNLDSKVGGNYNYLYRLFRKSNLKPFERDISTEIYQAGSTPCN
GVEGFNCYFPLQSVGFQPTNGVGYQPYRVVVLSFELLHAPATVCGPKKSTNLVKNKCVN
FNFNGLTGTGVLTESNKKFLPFQQFGRDAIDTTDAVRDPQTLEILDITPCS* [SEQ ID NO: 5]
pInt-Spike-RBD sequence
MRVQPTESIVRFPNITNLCPFGEVFNATRFASVYAWNRKRISNCVADVSVLYNSAS
FSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGFNCYFPLQS
VGFQPTNGVGYQPYRVVVLSFELLHAPATVCGPKKSTNLVKNKCVNFNFNGLTGTGVLT
ESNKKFLPFQQFGRDAIDTTDAVRDPQTLEILDITPCS* [SEQ ID NO: 6]
pLpp-OmpA-Spike-RBD sequence
MKATKLVLGAVILGSTLLAGCSSNAKIDQGINNNGPTHENQLGAGAFGGYQVNPY
VGFEMGYDWLGRMPYKGSVENGAYKAQGVQLTAKLGYPITDDLDIYTRLGGMVWRAD
TKSNVYGKNHDTGVSPVFAGGVEYAITPEIATRKLENLYFQGTRMRVQPTESIVRFPNITN
LCPFGEVFNATRFASVYAWNRKRISNCVADVSVLYNSASFSTFKCYGVSPTKLNDLCFTN
VYADSFVIRGDEVRQIAPGQTGKIADYNYKLPDDFTGCVIAWNSNNLDSKVGGNYNYLY
RLFRKSNLKPFERDISTEIYQAGSTPCNGVEGFNCYFPLQSVGFQPTNGVGYQPYRVVVLS
FELLHAPATVCGPKKSTNLVKNKCVNFNFNGLTGTGVLTESNKKFLPFQQFGRDAIDTTD
AVRDPQTLEILDITPCS* [SEQ ID NO: 7]

What is claimed is:

1. A genetically modified bacterium having an outer membrane, the bacterium comprising one or more anti-spike glycoprotein nanobodies on the outer membrane of the bacterium.

2. The genetically modified bacterium of claim 1, wherein one or more of the anti-spike glycoprotein nanobodies have been fused with Intimin.

3. The genetically modified bacterium of claim 1, wherein one or more of the anti-spike glycoprotein nanobodies have been fused with Lpp-OmpA.

4. The genetically modified bacterium of claim 1, wherein one or more of the anti-spike glycoprotein nanobodies further comprise one or more restriction sites.

5. The genetically modified bacterium of claim 1, wherein at least one anti-spike glycoprotein nanobody has a sequence of [SEQ ID NO: 1].

6. The genetically modified bacterium of claim 1, wherein at least one anti-spike glycoprotein nanobody has a sequence of [SEQ ID NO: 2].

7. The genetically modified bacterium of claim 1, wherein at least one anti-spike glycoprotein nanobody has a sequence of [SEQ ID NO: 3].

8. The genetically modified bacterium of claim 1, wherein at least one anti-spike glycoprotein nanobody has a sequence of [SEQ ID NO: 4].

9. The genetically modified bacterium of claim 1, wherein the genetically modified bacterium is a probiotic.

10. The genetically modified bacterium of claim 1, wherein the bacterium that is modified is E. coli.

11. The genetically modified bacterium of claim 1, wherein the bacterium that is modified is E. coli Nissle 1917.

12. A pharmaceutical composition comprising the genetically modified bacterium of claim 1 and a pharmaceutically acceptable excipient.

13. A genetically modified bacterium having an outer membrane, wherein the receptor-binding domain of the spike glycoprotein on SARS-COV-2 (Spike-RBD) is expressed on the outer membrane of bacterium.

14. The genetically modified bacterium of claim 13, wherein the Spike-RBD has been fused with Intimin.

15. The genetically modified bacterium of claim 13, wherein the Spike-RBD has been fused with Lpp-OmpA.

16. The genetically modified bacterium of claim 13, wherein the Spike-RBD has a sequence of [SEQ ID NO: 6].

17. The genetically modified bacterium of claim 13, wherein the Spike-RBD has a sequence of [SEQ ID NO: 7].

18. The genetically modified bacterium of claim 13, wherein the genetically modified bacterium is a probiotic.

19. The genetically modified bacterium of claim 13, wherein the bacterium that is modified is E. coli.

20. The genetically modified bacterium of claim 13, wherein the bacterium that is modified is E. coli Nissle 1917.

21. A pharmaceutical composition comprising the genetically modified bacterium of claim 13 and a pharmaceutically acceptable excipient.


SEQUENCE LISTING (7 sequences)

SEQ ID NO: 1 - protein, 812 aa, synthetic construct
MITHGCYTRTRHKHKLKKTLIMLSAGLLGLFFYVNQNSFANGENYFKLGSDSKLLTIIDSYQNRLFYTLKTGETVADLSKSQDINLSTIWSLNKHLYSSESEMMKAAPGQQIILPLKKLPFEYSALPLLGSAPLVAAGGVAGHTNKLTKMSPDVTKSNMTDDKALNYAAQQAASLGSQLQSRSLNGDYAKDTALGIAGNQASSQLQAWLQHYGTAEVNLQSGNNFDGSSLDFLLPFYDSEKMLAFGQVGARYIDSRFTANLGAGQRFFLPANMLGYNVFIDQDFSGDNTRLGIGGEYWRDYFKSSVNGYFRMSGWHESYNKKDYDERPANGFDIRFNGYLPSYPALGAKLIYEQYYGDNVALFNSDKLQSNPGAAVGVNYTPIPLVTMGIDYRHGTGNENDLLYSMQFRYQFDKSWSQQIEPQYVNELRFLSGSRYDLVQRNNNILEEYKKQDILSNIPPHDINGEHSTQKIQLIVKSKYGLDRIVWDDSALRSQGGQIQHSGSQSAQDYQAILPAYVQGGSNIYKVTARAYDRNGNSSNNVQLTIIVLSNGQVVDQVGVTDFTADKTSAKADNADTITYTAVKKNGVAQANVPVSFNIVSGTATLGANSAKDANGKATVTLKSSTPGQVVVSAKTAEMTSALNASAVIFFDGAPGKLENLYFQGTRQVQLVETGGGLVQPGGSLRLSCAASGFTFSSVYMNWVRQAPGKGPEWVSRISPNSGNIGYIDSVKGRFTISRDNAQNTLYLQMNNLKPEDTALYYCAIGLNLSSSSVRGQGTQVTVSSGSENLYFQGDYKDDDDKWSHPQFEKSR

SEQ ID NO: 2 - protein, 821 aa, synthetic construct
MITHGCYTRTRHKHKLKKTLIMLSAGLLGLFFYVNQNSFANGENYFKLGSDSKLLTIIDSYQNRLFYTLKTGETVADLSKSQDINLSTIWSLNKHLYSSESEMMKAAPGQQIILPLKKLPFEYSALPLLGSAPLVAAGGVAGHTNKLTKMSPDVTKSNMTDDKALNYAAQQAASLGSQLQSRSLNGDYAKDTALGIAGNQASSQLQAWLQHYGTAEVNLQSGNNFDGSSLDFLLPFYDSEKMLAFGQVGARYIDSRFTANLGAGQRFFLPANMLGYNVFIDQDFSGDNTRLGIGGEYWRDYFKSSVNGYFRMSGWHESYNKKDYDERPANGFDIRFNGYLPSYPALGAKLIYEQYYGDNVALFNSDKLQSNPGAAVGVNYTPIPLVTMGIDYRHGTGNENDLLYSMQFRYQFDKSWSQQIEPQYVNELRFLSGSRYDLVQRNNNILEEYKKQDILSNIPPHDINGEHSTQKIQLIVKSKYGLDRIVWDDSALRSQGGQIQHSGSQSAQDYQAILPAYVQGGSNIYKVTARAYDRNGNSSNNVQLTIIVLSNGQVVDQVGVTDFTADKTSAKADNADTITYTAVKKNGVAQANVPVSFNIVSGTATLGANSAKDANGKATVTLKSSTPGQVVVSAKTAEMTSALNASAVIFFDGAPGKLENLYFQGTRQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGSGSENLYFQGDYKDDDDKWSHPQFEKSR

SEQ ID NO: 3 - protein, 300 aa, synthetic construct
MKATKLVLGAVILGSTLLAGCSSNAKIDQGINNNGPTHENQLGAGAFGGYQVNPYVGFEMGYDWLGRMPYKGSVENGAYKAQGVQLTAKLGYPITDDLDIYTRLGGMVWRADTKSNVYGKNHDTGVSPVFAGGVEYAITPEIATRKLENLYFQGTRQVQLVETGGGLVQPGGSLRLSCAASGFTFSSVYMNWVRQAPGKGPEWVSRISPNSGNIGYTDSVKGRFTISRDNAKNTLYLQMNNKPEDTALYYCAIGLNLSSSSVRGQGTQVTVSSGSENLYFQGDYKDDDDKWSHPQFEKSR

SEQ ID NO: 4 - protein, 310 aa, synthetic construct
MKATKLVLGAVILGSTLLAGCSSNAKIDQGINNNGPTHENQLGAGAFGGYQVNPYVGFEMGYDWLGRMPYKGSVENGAYKAQGVQLTAKLGYPITDDLDIYTRLGGMVWRADTKSNVYGKNHDTGVSPVFAGGVEYAITPEIATRKLENLYFQGTRQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGSGSENLYFQGDYKDDDDKWSHPQFEKSR

SEQ ID NO: 5 - protein, 941 aa, synthetic construct
MITHGCYTRTRHKHKLKKTLIMLSAGLLGLFFYVNQNSFANGENYFKLGSDSKLLTIIDSYQNRLFYTLKTGETVADLSKSQDINLSTIWSLNKHLYSSESEMMKAAPGQQIILPLKKLPFEYSALPLLGSAPLVAAGGVAGHTNKLTKMSPDVTKSNMTDDKALNYAAQQAASLGSQLQSRSLNGDYAKDTALGIAGNQASSQLQAWLQHYGTAEVNLQSGNNFDGSSLDFLLPFYDSEKMLAFGQVGARYIDSRFTANLGAGQRFFLPANMLGYNVFIDQDFSGDNTRLGIGGEYWRDYFKSSVNGYFRMSGWHESYNKKDYDERPANGFDIRFNGYLPSYPALGAKLIYEQYYGDNVALFNSDKLQSNPGAAVGVNYTPIPLVTMGIDYRHGTGNENDLLYSMQFRYQFDKSWSQQIEPQYVNELRFLSGSRYDLVQRNNNILEEYKKQDILSNIPPHDINGEHSTQKIQLIVKSKYGLDRIVWDDSALRSQGGQIQHSGSQSAQDYQAILPAYVQGGSNIYKVTARAYDRNGNSSNNVQLTIIVLSNGQVVDQVGVTDFTADKTSAKADNADTITYTAVKKNGVAQANVPVSFNIVSGTATLGANSAKDANGKATVTLKSSTPGQVVVSAKTAEMTSALNASAVIFFDGAPGKLENLYFQGTRMRVQPTESIVRFPNITNLCPFGEVFNATRFASVYAWNRKRISNCVADVSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSVGFQPTNGVGYQPYRVVVLSFELLHAPATVCGPKKSTNLVKNKCVNFNFNGLTGTGVLTESNKKFLPFQQFGRDAIDTTDAVRDPQTLEILDITPCS

SEQ ID NO: 6 - protein, 274 aa, synthetic construct
MRVQPTESIVRFPNITNLCPFGEVFNATRFASVYAWNRKRISNCVADVSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSVGFQPTNGVGYQPYRVVVLSFELLHAPATVCGPKKSTNLVKNKCVNFNFNGLTGTGVLTESNKKFLPFQQFGRDAIDTTDAVRDPQTLEILDITPCS

SEQ ID NO: 7 - protein, 430 aa, synthetic construct
MKATKLVLGAVILGSTLLAGCSSNAKIDQGINNNGPTHENQLGAGAFGGYQVNPYVGFEMGYDWLGRMPYKGSVENGAYKAQGVQLTAKLGYPITDDLDIYTRLGGMVWRADTKSNVYGKNHDTGVSPVFAGGVEYAITPEIATRKLENLYFQGTRMRVQPTESIVRFPNITNLCPFGEVFNATRFASVYAWNRKRISNCVADVSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSVGFQPTNGVGYQPYRVVVLSFELLHAPATVCGPKKSTNLVKNKCVNFNFNGLTGTGVLTESNKKFLPFQQFGRDAIDTTDAVRDPQTLEILDITPCS